SEQUENCE LISTING (1 sequence)

SEQ ID NO: 1 - protein, 253 aa, Homo sapiens
SKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPT

(19) United States
(12) Patent Application Publication
(10) Pub. No.: US 2019/0008797 A1
(43) Pub. Date: Jan. 10, 2019

(54) ESTROGEN RECEPTOR ALPHA COLIGANDS, AND METHODS OF USE THEREOF

(71) Applicant: The Regents of the University of California, Oakland, CA (US)

(72) Inventor: Dale C. Leitman, Berkeley, CA (US)

(21) Appl. No.: 16/067,977

(22) PCT Filed: Jan. 24, 2017
(86) PCT No.: PCT/US2017/014717

§ 371 Date: Jul. 3, 2018

Related U.S. Application Data
(60) Provisional application No. 62/286,718, filed on Jan. 25, 2016.

(51) Int. Cl. A61K 31/12; A61K 9/00; A61K 31/565; A61P 35/00

(52) U.S. Cl. CPC A61K 31/12; A61K 9/0019; A61P 35/00; A61K 31/565; A61K 9/0053

(57) ABSTRACT

Provided herein is a coligand for the estrogen receptor (ER) a subunit, and methods of use thereof in treating conditions associated with ER signaling in an individual. The present ERα coligand may be a cell type-selective, allosteric modulator of ERα signaling. The ERα coligand, when administered to an individual, may modulate ER agonist-dependent signaling in a tissue-selective manner.

### CROSS-REFERENCE

This application claims the benefit of U.S. Provisional Patent Application No.

62/286,718, filed January 25, 2016, which application is incorporated herein by reference in its entirety.

### INTRODUCTION

Estrogens are steroid hormones synthesized mainly in the ovaries that bind to two nuclear hormone receptors, estrogen receptor (ER) a and ERβ. A single estrogen molecule binds to the ligand binding domain (LBD) occupying the binding site of each subunit of an ER dimer of ERα and/or ERβ. Binding of estrogen to ER triggers a number of events, including phosphorylation of the receptor, dimerization of the receptor, and binding of the receptor dimer to DNA containing nucleotide sequence motifs called hormone response elements. The DNA/receptor complex recruits other proteins, and regulates gene expression and cell function. Different ligands which bind to the same binding pocket of ER and other nuclear receptors can create different conformations, leading to the recruitment of distinct coregulators to alter gene expression and biological effects.

Coligands are molecules that bind to a receptor at a secondary site that is different from the ligand binding site on the receptor. Thus, a coligand and a ligand may bind simultaneously to the receptor. The biological activity of the ligand-bound receptor may be different in the presence and absence of the coligand bound to the secondary site.

### SUMMARY

Provided herein is a coligand for the estrogen receptor (ER) a subunit, and methods of use thereof in treating conditions associated with ER signaling in an individual. The present method may include administering a therapeutically effective amount of an ERα coligand to an individual, wherein the ERα coligand is a cell type-selective, allosteric modulator of ERα signaling, thereby reprogramming ER agonist-dependent signaling in a tissue-selective manner

In any embodiment, the reprogramming may include: suppressing ER agonist-dependent cell proliferation in breast and/or uterine tissue, relative to ER agonist-dependent cell proliferation in the absence of the ERα coligand; and/or increasing ER agonist-dependent transcription in bone, brain and/or adipose tissue, or other tissues, relative to ER agonist-dependent signaling in the absence of the ERα coligand.

In any embodiment, the ERα coligand may be a compound, or a pharmaceutically acceptable salt thereof, represented by the formula (I):

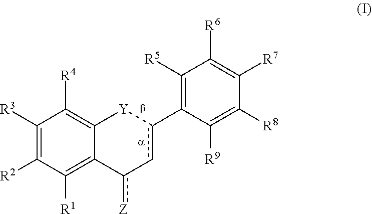

wherein α, β and γ are optional bonds, with the proviso that when β is absent, α is present; R¹, R², R³, R⁴, R⁵, R⁶, R⁷, R⁸, and R⁹ are independently hydrogen, hydroxyl, sulfhydryl, halo, alkyl, alkoxy, aryloxy, or arylalkyloxy, and may be heteroatom-containing and/or substituted; Y is hydrogen, hydroxyl, sulfhydryl, halo, alkyl, alkoxy, aryloxy, or arylalkyloxy, when β is absent, and O, when β is present; and Z is O, S or NR¹⁰, where R¹⁰ is hydrogen or alkyl. In some embodiments, the ERα coligand has a molecular weight in the range of 250 to 260 g/mol. In some embodiments, a and y are present. In some embodiments, the R⁵, R⁶, R⁷, R⁸, and R⁹ are hydrogen. In some embodiments, the ERα coligand is a chalcone derivative. In some embodiments, the the ERα coligand is a trihydroxychalcone. In some embodiments, the the ERα coligand is 2′,3′,4′-trihydroxychalcone (2′,3′,4′-THC).

In any embodiment, the ERα coligand may be administered at an amount sufficiently low to achieve a local concentration of the ERα coligand in a target tissue at which local concentration the coligand does not substantially compete with an ER agonist for binding to the estrogen binding site of ERα in the target tissue.

In any embodiment, the ERα coligand may modulate ERβ signaling. In some embodiments, the ERα coligand increases ERβ signaling relative to ERβ signaling in the absence of the ERα coligand.

In any embodiment, the administering may include parenterally or orally administering the ERα coligand to the patient. In any embodiment, the administering may include dermally or intranasally administering the ERα coligand to the patient.

In any embodiment, the method may further include co-administering a pharmaceutically effective amount of an ER agonist with the ERα coligand. In some embodiments, the ER agonist is estradiol (E2), or a derivative thereof. In some embodiments, the method does not comprise co-administering a progestin with the ER agonist. In some embodiments, the molar ratio of the ER agonist to the ERα coligand administered to the individual is 1:100 or less.

In any embodiment, allosteric modulation of ERα signaling by the ERα coligand may be inhibited by 2,2′,4′-THC at a concentration of 2,2′,4′-THC that does not inhibit ER agonist-dependent signaling.

In any embodiment, the ER-associated condition may include symptoms of menopause, side effects of menopausal hormone therapy and/or cancer. In some embodiments, the ER-associated condition comprises osteoporosis, breast cancer, endometrial cancer, colon cancer, pulmonary cancer, dementia, Alzheimer's disease, hot flashes, mood swings, insomnia, vaginal atrophy, vaginal dryness, dyspareunia, venous thromboembolism, gallbladder disease, endometriosis, metabolic syndrome, obesity and/or diabetes.

In any embodiment, the individual is a pre-, peri- or post-menopausal individual. For example, in some cases, the individual is a pre-menopausal female, e.g., a pre-menopausal human female. As another example, in some cases, the individual is a peri-menopausal female, e.g., a peri-menopausal human female. As another example, in some cases, the individual is a post-menopausal female, e.g., a post-menopausal human female.

Also provided herein is a pharmaceutical composition including a pharmaceutically effective amount of an ERα coligand in a pharmaceutically acceptable excipient, wherein the ERα coligand is a cell type-selective, allosteric modulator of ER signaling.

### BRIEF DESCRIPTION OF THE DRAWINGS

FIGS. 1a-1f are a collection of graphs and diagrams showing 2′,3′, 4′-trihydroxychalcone (2′,3′,4′-THC) synergizing estradiol (E2) induced transcriptional activity of estrogen response element (ERE)-tk luciferase in U2OS cells expressing ERα or ERβ, according to embodiments of the present disclosure.

FIGS. 2a-2f are a collection of graphs and diagrams showing 2′, 3′, 4′-THC behavior as a unique coagonist on gene expression in U2OS-ERα cells, according to embodiments of the present disclosure.

FIGS. 3a-3d are a collection of graphs and images showing 2′, 3′, 4′-THC binding to the surface of ERα by in silico modeling, according to embodiments of the present disclosure.

FIGS. 4a-4h are a collection of graphs and images showing 2′, 3′, 4′-THC blocking E2-induced MCF-7 cell proliferation by causing a G1 cell cycle arrest, according to embodiments of the present disclosure.

FIGS. 5a-5h are a collection of graphs and images showing 2′, 3′, 4′-THC acting as antagonist in the mouse uterus, according to embodiments of the present disclosure.

FIGS. 6a-6d are a collection of graphs showing the synergistic activation of E2-induced genes by 2′, 3′, 4′-THC being blocked by the ER antagonist ICI 182,780.

FIGS. 7a-7e are a collection of graphs showing specificity of synergistic stimulation of E2 activation of reporter genes for ER.

### DEFINITIONS

The nomenclature of certain compounds or substituents are used in their conventional sense, such as described in chemistry literature including but not limited to Loudon, Organic Chemistry, Fourth Edition, N.Y.: Oxford University Press, 2002, pp. 360-361, 1084-1085; Smith and March, March's Advanced Organic Chemistry: Reactions, Mechanisms, and Structure, Fifth Edition, Wiley-Interscience, 2001.

As used herein, the term “alkyl” by itself or as part of another substituent refers to a saturated branched or straight-chain monovalent hydrocarbon radical derived by the removal of one hydrogen atom from a single carbon atom of a parent alkane Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group contains from 1 to 20 carbon atoms. In other embodiments, an alkyl group contains from 1 to 10 carbon atoms. In still other embodiments, an alkyl group contains from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1 -en-2-yl, 2-methyl-prop-1-en-1 -yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Acyl” by itself or as part of another substituent refers to a radical —C(O)R³⁰, where R³⁰ is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, succinyl, and malonyl, and the like.

“Alkoxy” by itself or as part of another substituent refers to a radical −OR³¹ where R³¹ represents an alkyl or cycloalkyl group as defined herein. Representative examples include, but are not limited to, methoxy, ethoxy, propoxy, butoxy, cyclohexyloxy and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group contains from 6 to 12 carbon atoms. Examples of an aryl group are phenyl and naphthyl.

The term “aryloxy” as used herein refers to an aryl group bound through a single, terminal ether linkage, wherein “aryl” is as defined above. An “aryloxy” group may be represented as —O-aryl where aryl is as defined above. In certain embodiments, an aryloxy group contains 5 to 20 carbon atoms, e.g., 5 to 12 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain embodiments, an arylalkyl group is (C₇-C₃₀) arylalkyl, e.g., the alkanyl, alkenyl or alkynyl moiety of the arylalkyl group is (C₁-C₁₀) and the aryl moiety is (C₆-C₂₀). In certain embodiments, an arylalkyl group is (C₇-C₂₀) arylalkyl, e.g., the alkanyl, alkenyl or alkynyl moiety of the arylalkyl group is (C₁-C₈) and the aryl moiety is (C₆-C₁₂).

The term “arylalkyloxy” refers to an arylalkyl group bound through a single, terminal ether linkage. As above, an “arylalkyloxy” group may be represented as —O-Alk(Ar) wherein “Alk” is an alkyl group and “Ar” is an aryl substituent. Aralkyloxy substituents include, for example, benzyloxy, 2-phenoxy-ethyl, 3-phenoxy-propyl, 2-phenoxy-propyl, 2-methyl-3-phenoxypropyl, 2-ethyl-3-phenoxypropyl, 4-phenoxy-butyl, 3-phenoxy-butyl, 2-methyl-4-phenoxybutyl, 4-phenoxycyclohexyl, 4-benzyloxycyclohexyl, 4-phenoxy-cyclohexylmethyl, 2-(4-phenoxy-cyclohexyl)-ethyl, and the like.

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenyl-napthyl, binaphthyl, biphenyl-napthyl, and the like. When the number of carbon atoms in an arylaryl group are specified, the numbers refer to the carbon atoms comprising each aromatic ring. For example, (C₅-C₁₄) arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a (C₅-C₁₄) aromatic. In certain embodiments, each aromatic ring system of an arylaryl group is independently a (C₅-C₁₀) aromatic. In certain embodiments, each aromatic ring system is identical, e.g., biphenyl, triphenyl, binaphthyl, trinaphthyl, etc.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is (C₃-C₁₀) cycloalkyl. In certain embodiments, the cycloalkyl group is (C₃-C₇) cycloalkyl.

“Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR³⁷R³⁸—, .═N—N═, —N═N—, —N═N—NR³⁹R⁴⁰, —PR⁴¹—, —P(O)₂—, —POR⁴²—, —O—P(O)₂—, —S—O—, —S—(O)—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Aromatic Ring System” by itself or as part of another substituent, refers to an unsaturated cyclic or polycyclic ring system having a conjugated 7E electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Heteroaromatic Ring System” by itself or as part of another substituent, refers to an aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Substituted” refers to a group in which one or more hydrogen atoms are independently replaced with the same or different substituent(s). Typical substituents include, but are not limited to, alkylenedioxy (such as methylenedioxy), —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶² C(O)NR⁶⁰R⁶¹, —NR⁶²C(S)NR⁶⁰R⁶¹, NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —NR⁶²C(O)NR⁶⁰R⁶¹. In certain embodiments, substituents include —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O⁻. In certain embodiments, substituents include —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O) OR⁶⁰ , —C(O)O⁻, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group.

The terms “halo” and “halogen” are used in the conventional sense to refer to a chloro, bromo, fluoro or iodo substituent.

The compounds described herein can contain one or more chiral centers and/or double bonds and therefore, can exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

“Derivative” as used herein, may describe a variant compound that is based on the core structure of a parent compound. The variant compound may have the same type of pharmacological or physiological activity as the parent compound.

The term “about” as used herein when referring to a measurable value such as an amount, a temporal duration, and the like, is meant to encompass variations of ±20% or ±10%, e.g., ±5%, ±1%, or ±0.1% from the specified value, as such variations are appropriate to perform the disclosed methods.

“Substantially” as used herein, may be applied to modify any quantitative representation that could permissibly vary without resulting in a change in the basic function to which it is related.

An “individual” as used herein, may be any suitable animal amenable to the methods and techniques described herein, where in some cases, the individual may be a vertebrate animal, including a mammal, bird, reptile, amphibian, etc. The individual may be any suitable mammal, e.g., human, mouse, rat, cat, dog, pig, horse, cow, monkey, non-human primate, etc. In some cases, the individual is a patient, e.g., an individual in need of treatment for a disease. In some cases, the individual is a human. In some cases, the individual is a human female.

A “condition” as used herein, refers to a deviation from a desirable physiological status of an individual. The deviation may be a change in hormone levels, metabolism and/or tissue function, etc. The deviation may be spontaneous, idiopathic, age-related, sex-specific, and/or pharmacologically induced, etc. The condition in some cases may be a disease, such as cancer, diabetes, osteoporosis, gallbladder disease, etc. The condition in some cases may be a secondary condition induced by treatment of a primary condition, e.g., a side effect of treating a primary condition or a disease.

As used herein, the terms “treat,” “treatment,” “treating,” and the like, refer to obtaining a desired pharmacologic and/or physiologic effect. The effect may be prophylactic in terms of completely or partially preventing a health condition, disease or symptoms thereof and/or may be therapeutic in terms of a partial or complete cure for a health condition, disease and/or adverse effect attributable to the health condition or disease. “Treatment,” as used herein, covers any treatment of a health condition or disease in a mammal, particularly in a human, and includes: (a) preventing the health condition or disease from occurring in a subject which may be predisposed to the health condition or disease but has not yet been diagnosed as having it; (b) inhibiting the health condition or disease, i.e., arresting its development; and (c) relieving the health condition or disease, e.g., causing regression of the disease, e.g., to completely or partially remove symptoms of the health condition or disease.

A “therapeutically effective amount” or “efficacious amount” means the amount of an agent that, when administered to a mammal or other subject for treating a disease, is sufficient to effect such treatment for the disease or condition. The “therapeutically effective amount” will vary depending on agent, the disease or condition and its severity and the age, weight, etc., of the subject to be treated.

By “pharmaceutically acceptable” is meant a material that is not biologically or otherwise undesirable, i.e., the material may be incorporated into a pharmaceutical composition administered to an individual without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

“Co-administer”, as used herein, may refer to administering two or more therapeutic agents to an individual to treat a disease. The two or more therapeutic agents may be administered with dosage schedules that are independent of one another (e.g., at different frequencies or intervals of administration). In some cases, two or more therapeutic agents may be administered at the same time, and in some cases, two or more therapeutic agents may be administered at different times (e.g., one before another, in alternating sequences, etc.)

The terms “cancer, “neoplasm,” and “tumor” are used interchangeably herein to refer to cells which exhibit relatively autonomous growth, so that they exhibit an aberrant growth phenotype characterized by a significant loss of control of cell proliferation. Cells of interest that may exhibit uncontrolled proliferation include precancerous, malignant, pre-metastatic, metastatic, and non-metastatic cells, as well as carcinoma in situ. Cancer also refers to the pathological condition caused by the uncontrolled proliferation of cells.

“Selective” as used herein, may be used to describe an effect induced or an outcome observed in a set of elements containing two or more subsets, where the effect is induced or outcome is differentially observed within a first subset set over a second, distinct subset. The effect or outcome may be different in magnitude, temporal pattern and/or spatial pattern, etc., between the first subset and the second subset. The difference may be quantitative, or qualitative.

“Allosteric” as used herein may be used to describe an interaction between a compound and a receptor at a site on the receptor that is biochemically and/or physically distinct from the agonist/ligand-binding site, e.g., at a secondary site of the receptor.

A “modulator” as used herein, may refer to an agent that causes a measurable and/or physiologically relevant change. The change may be qualitative and/or quantitative. The change may be a change in temporal pattern, a change in magnitude, a change in sign, etc.

An “agonist” as used herein, refers to a compound that induces one or more physiologically relevant activities of a cellular receptor when the compound binds to a ligand binding-site of the receptor. “Agonist-dependent” as used herein, may be used to describe a cellular signaling event that is induced by the agonist, e.g., after the agonist binds to its receptor.

“Reprogram” as used herein, refers to selectively or specifically perturbing the course of one or more sequence of events such that one or more outcomes of the sequence of events deviate from their respective unperturbed outcomes.

Before the present disclosure is further described, it is to be understood that the disclosed subject matter is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter. The upper and lower limits of these smaller ranges may independently be included in the smaller ranges, and are also encompassed within the disclosed subject matter, subject to any specifically excluded limit in the stated range. Where the stated range includes one or both of the limits, ranges excluding either or both of those included limits are also included in the disclosed subject matter.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an ER agonist” includes a plurality of such ER agonists and reference to “the target tissue” includes reference to one or more target tissues and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination. All combinations of the embodiments pertaining to the disclosure are specifically embraced by the disclosed subject matter and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the disclosed subject matter is not entitled to antedate such publication by virtue of prior invention. Further, the dates of publication provided may be different from the actual publication dates which may need to be independently confirmed.

### DETAILED DESCRIPTION

As summarized above, aspects of the present disclosure include an active agent that functions as a coligand for the estrogen receptor (ER) a subunit, where the active agent may be a cell type-selective, allosteric modulator of ERα signaling. Active agent compounds of the present disclosure can bind to ERα at a site that is different from the estrogen binding site of ERα, and upon binding, the present active agent modulates estrogen-induced signaling mediated by the ER. Without wishing to be bound to theory, an ERα coligand of the present disclosure may induce a conformational change in the estrogen-bound ERα of an ER dimer, alter the set of coregulators recruited to the ER and bound to DNA, and generate distinct biological responses (e.g., transcriptionally regulate distinct sets of genes), compared to ER bound to estrogen in the absence of the ERα coligand. The specific set of coregulators and their susceptibility to the conformational change induced by the ERα coligand may vary depending on the cell type in which the ER resides. Thus, the outcome of ERα coligand modulation of estrogen-dependent signaling through the ER may depend on the cell type.

Further aspects of the present ERα coligand are now described.

### ERα Coligands

A ERα coligand of the present disclosure includes any suitable active agent, e.g., compound, that modulates ER agonist signaling mediated by ERα by an allosteric mechanism, and where the manner of modulation depends on the cell type in which ER signaling is induced by the ER agonist, as described further below. Any suitable method may be used to identify the ERα coligand, as described further below.

In some embodiments, a ERα coligand of the present disclosure is a compound, or a salt thereof, represented by the formula (I):

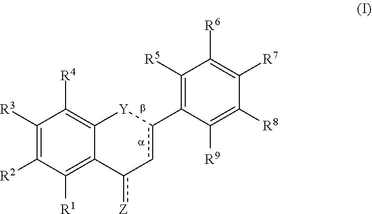

where α, β and γ are optional bonds, with the proviso that when β is absent, a is present; R¹, R², R³, R⁴, R⁵, R⁶, R⁷, R⁸, and R⁹ are independently hydrogen, hydroxyl, sulfhydryl, halo, alkyl, alkoxy, aryloxy, or arylalkyloxy, and may be heteroatom-containing and/or substituted; Y is hydrogen, hydroxyl, sulfhydryl, halo, alkyl, alkoxy, aryloxy, or arylalkyloxy, when β is absent, and O, when β is present; and Z is O, S or NR¹⁰, where R¹⁰ is hydrogen or alkyl. In some embodiments, Z is O and γ is present. In some embodiments, Y is O and β is present. In some embodiments, β is absent and α is present. In some embodiments, Z is O, and α and γ are present. In certain embodiments, any three of R¹, R², R³, R⁴, and Y are hydroxyl and the rest is hydrogen, and R⁵, R⁶, R⁷, R⁸, and R⁹ are hydrogen.

In some embodiments, the ERα coligand is a chalcone derivative, represented by the formula (II):

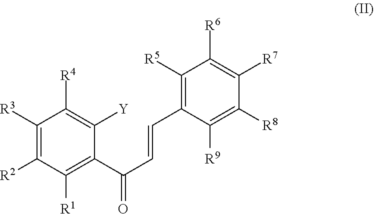

where R¹, R², R³, R⁴, R⁵, R⁶, R⁷, R⁸, R⁹, and Y are independently hydrogen, hydroxyl, sulfhydryl, halo, alkyl, alkoxy, aryloxy, or arylalkyloxy, and may be heteroatom-containing and/or substituted. In some embodiments, the ERα coligand is a trihydroxychalcone, where any three of R¹, R², R³, R⁴, R⁵, R⁶, R⁷, R⁸, R⁹, and Y are hydroxyl and the rest are hydrogen. In certain embodiments, any three of R¹, R², R³, R⁴, and Y are hydroxyl and the rest is hydrogen, and R⁵, R⁶, R⁷, R⁸, and R⁹ are hydrogen.

In some embodiments the chalcone derivative is 2′, 2,3,6′-tetramethoxychalcone, 2,2′, 4′, 6′-tetramethoxychalcone, 2,2′, 5′-trihydroxychalcone, 2,2′-dihydroxy-3-methoxychalcone, 2,2′-dihydroxy-4′, 6′-dimethoxychalcone, 2,2′-dihydroxychalcone, 2′, 3,4,5,6′-pentamethoxychalcone, 2′, 3′, 4′-trihydroxychalcone, 2,3-dimethoxy-2′-hydroxychalcone, 2′, 4,4′, 6′-tetramethoxychalcone, 2′, 4′, 6′-trimethoxychalcone, 2′, 4,6′-trimethoxychalcone, 2′, 4′-dichloro-3,4-dimethoxy-5′-fluorochalcone, 2′, 4′-dichloro-5′-fluoro-4-methoxychalcone, 2′, 4′-dihydroxy-2,3-dimethoxychalcone, 2′, 4′-dihydroxy-2-methoxychalcone, 2′, 4′-dihydroxy-3,4-dimethoxychalcone, 2′, 4-dihydroxy-4′, 6′-dimethoxychalcone, 2′, 4′-dihydroxy-4-methoxychalcone, 2′, 4′-dihydroxychalcone, 2,4-dimethoxy-2′-hydroxy-5′-methylchalcone, 2,4′-dimethoxy-2′-hydroxychalcone, 2,4-dimethoxy-2′-hydroxychalcone, 2′, 4′-dimethoxychalcone, 2′, 5′-dihydroxy-4-methoxychalcone, 2′, 5′-dihydroxychalcone, 2,5-dimethoxy-2′-hydroxy-5′-methylchalcone2,5′-dimethoxy-2′-hydroxychalcone, 2,5-dimethoxy-2′-hydroxychalcone, 2,5-dimethoxy-5′-fluoro-2′-hydroxychalcone, 2,6-dichloro-2′-hydroxychalcone, 2′, 6′-dihydroxy-4,4′-dimethoxychalcone, 2′, 6′-dihydroxy-4,4′-dimethoxydihydrochalcone, 2′, 6′-dihydroxy-4-methoxychalcone-4′-o-neohesperidoside, 2,6′-dimethoxy-2′-hydroxychalcone, 2′, 6′-dimethoxy-4-fluorochalcone, 2′, 6′-dimethoxychalcone, 2-chloro-2′, 4′, 6′-trimethoxychalcone, 2-chloro-4′, 6′-dimethoxy-2′-hydroxychalcone, 2′-hydroxy-2,3,4′, 6′-tetramethoxychalcone, 2′-hydroxy-2,3,4′-trimethoxychalcone, 2′-hydroxy-2,3,5′-trimethoxychalcone, 2′-hydroxy-2,3,6′-trimethoxychalcone, 2′-hydroxy-2,4,4′, 5-tetramethoxychalcone, 2′-hydroxy-2,4,4′, 6′-tetramethoxychalcone, 2′-hydroxy-2,4,4′-trimethoxychalcone, 2′-hydroxy-2,4,5,4′, 6′-pentamethoxychalcone, 2′-hydroxy-2,4,5,5′-tetramethoxychalcone, 2′-hydroxy-2,4′, 5,6′-tetramethoxychalcone, 2′-hydroxy-2,4,5,6′-tetramethoxychalcone, 2′-hydroxy-2,4′, 5-trimethoxychalcone, 2′-hydroxy-2,4,5′-trimethoxychalcone, 2′-hydroxy-2,4,5-trimethoxychalcone, 2′-hydroxy-2,4′, 6′-trimethoxychalcone, 2′-hydroxy-2,4,6′-trimethoxychalcone, 2-hydroxy-2′, 4′, 6′-trimethoxychalcone, 2′-hydroxy-2,5,6′-trimethoxychalcone, 2′-hydroxy-2-methoxychalcone, 2′-hydroxy-3,3′, 4′-trimethoxychalcone, 2′-hydroxy-3,4,4′, 5′-tetramethoxychalcone, 2′-hydroxy-3,4,4′, 6′-tetramethoxychalcone, 2′-hydroxy-3′, 4′, 4-trimethoxychalcone, 2′-hydroxy-3,4,4′-trimethoxychalcone, 2′-hydroxy-3,4,5,6′-tetramethoxychalcone, 2′-hydroxy-3′, 4,5′-trichlorochalcone, 2′-hydroxy-3,4,5′-trimethoxychalcone, 2′-hydroxy-3,4,5-trimethoxychalcone, 2′-hydroxy-3,4,6′-trimethoxychalcone, 2′-hydroxy-3′, 4′-benzochalcone, 2′-hydroxy-3-methoxychalcone, 2-hydroxy-3-methoxychalcone, 2′-hydroxy-4,4′, 6′-trimethoxychalcone, 2′-hydroxy-4-methoxy-5′-methylchalcone, 2′-hydroxy-4′-methoxychalcone, 2′-hydroxy-4-methoxychalcone, 2′-hydroxy-4-methylchalcone, 2′-hydroxy-5′-methyl-2-methoxychalcone, 2′-hydroxy-5′-methyl-3,4-methylenedioxychalcone, 2′-hydroxy-5′-methylchalcone, 2′-hydroxy-6′-methoxychalcone, 2′-hydroxy-6′-methyl-3,4-methylenedioxychalcone, 2′-hydroxychalcone, 2-hydroxychalcone, 3,2′-dihydroxy-4,4′, 6′-trimethoxychalcone, 3,2′-dihydroxychalcone, 3,4,2′, 4′, 6′-pentahydroxychalcone with hplc, 3,4,2′, 4′, 6′-pentamethoxychalcone, 3,4,2′, 5′-tetramethoxychalcone, 3,4-dimethoxy-2′-hydroxy-5′-methylchalcone, 3′, 4′-dimethoxy-2′-hydroxychalcone, 3,4′-dimethoxy-2′-hydroxychalcone, 3,4′-dimethoxy-2-hydroxychalcone, 3,4-dimethoxy-2′-hydroxychalcone, 3,4-dimethoxychalcone, 3′, 5′-dichloro-2,5-dimethoxy-4′-hydroxychalcone, 3′, 5′-dichloro-2′-hydroxy-3,4,5-trimethoxychalcone, 3′, 5′-dichloro-2′-hydroxy-4-methoxychalcone, 3,6′-dimethoxy-2′-hydroxychalcone, 3-bromo-2′, 4′, 6′-trimethoxychalcone, 3-bromo-4′, 6′-dimethoxy-2′-hydroxychalcone, 3′-bromo-5′-chloro-2′-hydroxy-3,4,5-trimethoxychalcone, 3′-bromo-5′-chloro-2′-hydroxy-4-methoxychalcone, 3′-bromo-5′-chloro-2′-hydroxychalcone, 3′-chloro-2′-hydroxy-4-methoxychalcone, 3′-chloro-2′-hydroxy-4-methylchalcone, 3′-chloro-2′-hydroxychalcone, 3′-chloro-4-fluoro-2′-hydroxychalcone, 3-hydroxy-2′, 4,4′, 6′-tetramethoxychalcone, 3-methoxy-4,2′, 5′-trihydroxychalcone, 3-nitro-2′, 4′, 6′-trimethoxychalcone, 4,2′, 4′-trihydroxychalcone, 4,2′, 5′-trihydroxychalcone, 4,2′, 5′-trimethoxychalcone, 4,2′-dihydroxy-3,4′, 6′-trimethoxychalcone, 4,2′-dihydroxy-3-methoxy-5′-methylchalcone, 4,2′-dihydroxy-3-methoxychalcone, 4,2′-dihydroxychalcone, 4,4′-dimethoxy-2′-hydroxychalcone, 4,4′-dimethoxychalcone, 4,5′-dichloro-2′-hydroxy-4′-methylchalcone, 4,5′-dichloro-2′-hydroxychalcone, 4′, 6′-dimethoxy-2′-hydroxy-3-nitrochalcone, 4′, 6′-dimethoxy-2′-hydroxy-4-methylchalcone, 4′, 6′-dimethoxy-2′-hydroxychalcone, 4,6′-dimethoxy-2′-hydroxychalcone, 4′, 6′-dimethoxy-4-dimethylamino-2′-hydroxychalcone, 4′, 6′-dimethoxy-4-fluoro-2′-hydroxychalcone, 4-benzyloxy-2′, 3,4′, 6′-tetramethoxychalcone, 4-benzyloxy-2′-hydroxy-3,4′, 6′-trimethoxychalcone, 4-chloro-2′, 3′, 4′-trimethoxychalcone, 4-chloro-2′, 4′, 6′-trimethoxychalcone, 4′-chloro-2,5-dimethoxychalcone, 4-chloro-2′, 5′-dimethoxychalcone, 4-chloro-2′, 6′-dimethoxychalcone, 4′-chloro-2-hydroxy-3-methoxychalcone, 4-chloro-2′-hydroxy-5′-methylchalcone, 4-chloro-2′-hydroxychalcone, 4′-chloro-3,4,5-trimethoxychalcone, 4′-chloro-3,4-dimethoxychalcone, 4-chloro-4′, 6′-dimethoxy-2′-hydroxychalcone, 4-chloro-4′-fluorochalcone, 4′-chloro-4-methoxychalcone, 4-chloro-5′-fluoro-2′-hydroxychalcone, 4′-chlorochalcone, 4-chlorochalcone, 4-deoxyphloridzin, 4-dimethylamino-2′, 4′, 6′-trimethoxychalcone, 4-fluoro-2′, 4′, 6′-trimethoxychalcone, 4-hydroxy-2′, 3,4′, 6′-tetramethoxychalcone, 4′-hydroxy-2,3′, 5,5′-tetramethoxychalcone, 4-hydroxy-2′, 4′, 6′-trimethoxychalcone, 4′-hydroxy-2′-methyl-3,4,5-trimethoxychalcone, 4′-hydroxy-4-methoxy-2′-methylchalcone, 4′-hydroxychalcone, 4-hydroxychalcone, 4-isopropyl-4′-methylchalcone, 4-methoxy-4′-(methylthio)chalcone, 4′-methoxychalcone, 4-methoxychalcone, 4-methyl-2′, 4′, 6′-trimethoxychalcone, 4′-methyl-3,4,5-trimethoxychalcone, 5-bromo-2,2′-dihydroxy-4′, 6′-dimethoxychalcone, 5′-bromo-2,5-dimethoxy-2′-hydroxychalcone, 5-bromo-2-hydroxy-2′, 4′, 6′-trimethoxychalcone, 5′-bromo-2′-hydroxy-3,4,5-trimethoxychalcone, 5′-bromo-2′-hydroxy-4-methoxychalcone, 5′-bromo-3,4-dimethoxy-2′-hydroxychalcone, 5′-bromo-3′-chloro-2,5-dimethoxy-2′-hydroxychalcone, 5′-bromo-3′-chloro-2′-hydroxy-3,4,5-trimethoxychalcone, 5′-bromo-3′-chloro-2′-hydroxy-4-methoxychalcone, 5′-bromo-3′-chloro-2′-hydroxychalcone, 5′-bromo-4,2′-dihydroxy-3-methoxychalcone, 5′-bromo-4-chloro-2′-hydroxychalcone, 5′-chloro-2,5-dimethoxy-2′-hydroxy-4-methylchalcone, 5′-chloro-2,5-dimethoxy-2′-hydroxychalcone, 5′-chloro-2′-hydroxy-3,4,5-trimethoxychalcone, 5′-chloro-2′-hydroxy-4-methoxy-4′-methylchalcone, 5′-chloro-2′-hydroxy-4′-methoxychalcone, 5′-chloro-2′-hydroxy-4′-methyl-3,4,5-trimethoxychalcone, 5′-chloro-2′-hydroxy-4′-methylchalcone, 5′-chloro-2′-hydroxy-4-methylchalcone, 5′-chloro-2′-hydroxychalcone, 5′-chloro-3,4-dimethoxy-2′-hydroxychalcone, 5′-chloro-4,2′-dihydroxy-3-methoxy-4′-methylchalcone, 5′-chloro-4,2′-dihydroxy-3-methoxychalcone, 5′-fluoro-2′-hydroxy-4-methoxychalcone, 5′-fluoro-2′-hydroxy-4-methylchalcone, butein, homobutein, or phloretin.

In certain embodiments, the ERα coligand is 2′, 3′, 4′-trihydroxychalcone, represented by the formula (III):

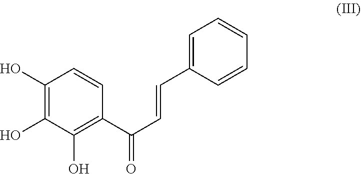 [SMILES: O=C(/C=C/c1ccccc1)c1ccc(O)c(O)c1O]

In some embodiments, an ERα coligand of the present disclosure is a flavonoid derivative, including, but not limited to, anthoxanthins, flavanols, flavanones, flavans, and anthocyanidins.

The ERα coligand of the present disclosure may have any suitable molecular weight. In some embodiments, the ERα coligand has a molecular weight of 250 g/mol or more, e.g., 251 g/mol or more, 252 g/mol or more, 253 g/mol or more, 254 g/mol or more, including 255 g/mol or more, and has a molecular weight of 260 g/mol or less, e.g., 259 g/mol or less, 258 g/mol or less, including 257 g/mol or less. In certain embodiments, the ERα coligand has a molecular weight in the range of 250 to 260 g/mol, e.g., 252 to 259 g/mol, 254 to 258 g/mol, including 255 to 257 g/mol.

A ERα coligand of the present disclosure may allosterically modulate ER agonist-dependent, ERα-mediated signaling in a cell type-selective manner An effective amount of the ERα coligand to achieve allosteric modulation of ER signaling, e.g., ER agonist-dependent, ERα-mediated signaling, may include a range of concentration of the ERα coligand at which the ERα coligand does not compete for binding with the ER agonist, e.g., estradiol (E2), for the ligand binding site.

The cell type-selective modulation of ER agonist-dependent ER signaling by the ERα coligand may include any suitable change, when compared to, e.g., ER agonist-dependent ER signaling in the absence of the ERα coligand, of the ER agonist-dependent ER signaling in any suitable set of cell types. In some cases, the change is a suppression or reduction in ER signaling, e.g., ER agonist-dependent, ERα-mediated signaling in a cell type-selective manner In some cases, the change is an enhancement or increase in ER signaling, e.g., ER agonist-dependent, ERα-mediated signaling in a cell type-selective manner In some cases, the change is a suppression or reduction in ER signaling, e.g., ER agonist-dependent, ERα-mediated signaling in cells of a first cell type, and an enhancement or increase in ER signaling, e.g., ER agonist-dependent, ERα-mediated signaling in cells of a second cell type, relative to ER agonist-dependent ER signaling in the absence of the ERα coligand in each cell type.

Signaling may be measured using any suitable physiologically relevant parameter, such as, but not limited to, expression of endogenous genes regulated by ER signaling, expression of exogenous reporter constructs configured to be regulated by ER signaling; localization of ER to transcriptional control elements on DNA; and/or any suitable cellular phenotype, such as cell proliferation and/or cell differentiation.

Thus, in some embodiments, where the ERα coligand suppresses or reduces ER signaling, if ER signaling, e.g., ER agonist-dependent, ERα-mediated signaling, normally induces expression of a target gene and/or promotes proliferation and/or differentiation of a target cell, allosteric modulation of ER signaling by the ERα coligand may reduce target gene expression and/or target cell proliferation and/or differentiation in the presence of an ER agonist, e.g., E2, and the ERα coligand, compared to the level of target gene expression and/or target cell proliferation and/or differentiation in an appropriate control, e.g., in the presence of a comparable amount of the ER agonist but in the absence of the ERα coligand. Similarly, in some embodiments, if ER signaling, e.g., ER agonist-dependent, ERα-mediated signaling, normally reduces target gene expression and/or suppresses target cell proliferation and/or differentiation, allosteric modulation of ER signaling by the ERα coligand may enhance target gene expression and/or target cell proliferation and/or differentiation in the presence of an ER agonist and the ERα coligand, compared to the level of target gene expression and/or target cell proliferation and/or differentiation in an appropriate control. The ERα coligand may suppress or reduce ER signaling, e.g., ER agonist-dependent, ERα-mediated signaling, in a cell type-selective manner, compared to ER signaling, e.g., ER agonist-dependent ERα-mediated signaling, in the absence of the ERα coligand, by 10% or more, e.g., 20% or more, 30% or more, 40% or more, 50% or more, 60% or more, 70% or more, 80% or more, 90% or more, and up to about 100%, where 100% reduction corresponds to the level of ER signaling in the absence of the ER agonist or ERα coligand.

In some embodiments, the ERα coligand may lower the effective concentration of the ligand. Thus, in some cases, the ERα coligand may shift the dose-response curve of ER signaling by an ER agonist, e.g., E2, to the right such that a higher concentration of the ER agonist is required in the presence of the ERα coligand to achieve the same level of ER signaling, e.g., ER agonist-dependent, ERα-mediated signaling, as in the absence of the ERα coligand.

In some embodiments, where the ERα coligand enhances or increases ER signaling, if ER signaling, e.g., ER agonist-dependent, ERα-mediated signaling, normally induces expression of a target gene and/or promotes proliferation and/or differentiation of a target cell, allosteric modulation of ER signaling by the ERα coligand may increase target gene expression and/or target cell proliferation and/or differentiation in the presence of an ER agonist and the ERα coligand, compared to the level of target gene expression and/or target cell proliferation and/or differentiation in an appropriate control, e.g., in the presence of a comparable amount of the ER agonist but in the absence of the ERα coligand Similarly, in some embodiments, if ER signaling, e.g., ER agonist-dependent, ERα-mediated signaling, normally reduces target gene expression and/or suppresses target cell proliferation and/or differentiation, allosteric modulation of ER signaling by the ERα coligand may further reduce target gene expression and/or target cell proliferation and/or differentiation in the presence of an ER agonist and the ERα coligand, compared to the level of target gene expression and/or target cell proliferation and/or differentiation in an appropriate control. The ERα coligand may enhance or increase ER signaling, e.g., ER agonist-dependent, ERα-mediated signaling, in a cell type-selective manner, compared to ER signaling, e.g., ER agonist-dependent ERα-mediated signaling, in the absence of the ERα coligand, by 1.5 fold or more, e.g., 2 fold or more, 2.5 fold or more, 3 fold or more, 4 fold or more, 5 fold or more, 10 fold or more, including 15 fold or more, and in some cases, by 100 fold or less, e.g., 50 fold or less, 30 fold or less, 20 fold or less, 10 fold or less, including 3 fold or less. In some embodiments, the ERα coligand enhances or increases ER signaling, e.g., ER agonist-dependent, ERα-mediated signaling, in a cell type-selective manner, compared to ER signaling, e.g., ER agonist-dependent ERα-mediated signaling, in the absence of the ERα coligand, by 1.5 to 100 fold, e.g., 2 to 50 fold, 2 to 30 fold, 2 to 20 fold, including 2 to 10 fold.

In some embodiments, the ERα coligand may raise the effective concentration of the ER. Thus, in some cases, the ERα coligand may shift the dose-response curve of ER signaling by an ER agonist, e.g., E2, to the left such that a lower concentration of the ER agonist is required in the presence of the ERα coligand to achieve the same level of ER signaling, e.g., ER agonist-dependent, ERα-mediated signaling, as in the absence of the ERα coligand.

In modulating ER signaling allosterically, the present ERα coligand may bind to ERα at a secondary site that is biochemically distinct from the ERα ligand binding site, i.e. biochemically distinct from the binding site for estradiol. Thus the ERα coligand may not compete with estradiol for binding to ERα-expressing cells at a concentration of the ERα coligand sufficient for cell-type selective modulation of ERα signaling. The allosteric modulation of ERα signaling by the ERα coligand may be suppressed by a moiety that binds to the same secondary site as the ERα coligand, but does not cause allosteric modulation of ERα signaling. Thus in some cases, the allosteric modulation of ERα signaling by the ERα coligand is inhibited by a compound represented by the formula (III):

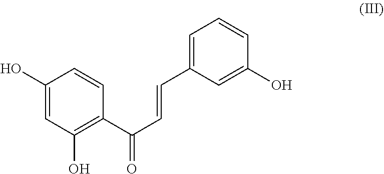 [SMILES: O=C(/C=C/c1cccc(O)c1)c1ccc(O)cc1O]

at a concentration of the compound that does not inhibit ER agonist-dependent signaling on its own. The inhibitor of the ERα coligand may be 2,2′, 4′-trihydroxychalcone (2,2′, 4′-THC).

The cell types in which the ERα coligand allosterically modulates ER signaling, e.g., ER agonist-dependent, ERα-mediated signaling, may be any suitable cell types where ERα is functionally expressed (endogenously and/or transgenically). The cell types may be primary cells or cell lines, e.g., cell lines derived from primary cells, cell lines, such as cancer cell lines, obtained from clinical samples, etc. Cell types of interest include, but are not limited to, bone cells, breast tissue cells, endometrial cells, adipose tissue cells, ovarian stromal cells, central nervous system cells (e.g., brain cells, such as, but not limited to, hypothalamic neurons), epithelial cells, kidney cells, cardiac myocytes, prostate tissue cells, endothelial cells, etc. In some cases, the cell types may be tumor cells or tumor-derived cells, such as, but not limited to, osteosarcoma cells, breast cancer cells, uterine carcinoma cells, etc.

In certain embodiments, an ERα coligand of the present disclosure differentially modulates ER signaling, e.g., ER agonist-dependent, ERα-mediated signaling, between two or more different (i.e., non-overlapping) sets of cell types, e.g., 3 or more different sets of cell types, including 4 or more different sets of cell types, and between 6 or fewer different sets of cell types, e.g., 5 or fewer different sets of cell types, including 4 or fewer different sets of cell types, when cells of the different sets are contacted with the ERα coligand at similar concentrations. In certain embodiments, an ERα coligand of the present disclosure differentially modulates ER signaling, e.g., ER agonist-dependent, ERα-mediated signaling, between 2 to 6 different (i.e., non-overlapping) sets of cell types, e.g., 2 to 5 different sets of cell types, including 2 to 4 different sets of cell types. The ER signaling may be measured in each set using the same or different parameters, as described above. Where the parameters measured are different between any two different sets of cell types, measurements of ER signaling may be normalized to provide an appropriate comparison of the effect of the ERα coligand on the two sets.

In some embodiments, the ERα coligand differentially modulates ER signaling, e.g., ER agonist-dependent, ERα-mediated signaling, between a first set of cell types including bone cells (such as osteosarcoma cells), a second set of cell types including breast tissue cells (such as breast cancer cells) and/or endometrial cells (such as uterine carcinoma cells), and a third set of cell types including adipose tissue cells. In some embodiments, ER signaling, e.g., ER agonist-dependent, ERα-mediated signaling, is increased in cells of the first set of cell types, is reduced or inhibited in cells of the second set of cell types, and is substantially unchanged in cells of the third set of cell types, by the presence of the ERα coligand in an effective amount, each compared to an appropriate control, e.g., compared to ER agonist-dependent ER signaling in the absence of the ERα coligand.

In certain embodiments, an ERα coligand of the present disclosure enhances ER agonist-dependent, ERα-mediated gene expression in bone cells (such as osteosarcoma cells), compared to an appropriate control, e.g., compared to ER agonist-dependent ER-mediated gene expression of bone cells in the absence of the ERα coligand. In certain embodiments, the ERα coligand suppresses or inhibits ER agonist-dependent, ERα-mediated gene expression in breast tissue cells (such as breast cancer cells), compared to an appropriate control. In certain embodiments, the ERα coligand suppresses or inhibits ER agonist-dependent, ERα-mediated cell proliferation in breast tissue cells (such as breast cancer cells), compared to an appropriate control. In certain embodiments, the ERα coligand suppresses or inhibits ER agonist-dependent, ERα-mediated cell proliferation in endometrial cells (such as uterine carcinoma cells), compared to an appropriate control. In certain embodiments, the ERα coligand does not substantially alter ER agonist-dependent, ERα-mediated cell proliferation and/or differentiation in adipose tissue cells (such as gonadal adipose tissue cells), compared to an appropriate control.

The present ERα coligand may, in some cases, modulate ER agonist-dependent ERβ-mediated signaling. Thus in some cases, contacting a cell functionally expressing an ER receptor containing one or two ERβ subunits, with an ER agonist and the ERα coligand increases or decreases ER signaling compared to the ER signaling in the absence of the ERα coligand. In some embodiments, the ERα coligand increases ER agonist-dependent ERI3-mediated signaling by 1.5 fold or more, e.g., 2 fold or more, 2.5 fold or more, 3 fold or more, 4 fold or more, 5 fold or more, 10 fold or more, including 15 fold or more, and in some cases, by 100 fold or less, e.g., 50 fold or less, 30 fold or less, 20 fold or less, 10 fold or less, including 3 fold or less. In some embodiments, the ERα coligand increases ER agonist-dependent ERβ-mediated signaling by 1.5 to 100 fold, e.g., 2 to 50 fold, 2 to 30 fold, 2 to 20 fold, including 2 to 10 fold.

### Compositions and Pharmaceutical Formulations

Also provided herein is a composition, e.g., a pharmaceutical composition, that includes a pharmaceutically effective amount of an ERα coligand, as described above, in a pharmaceutically acceptable excipient. As used herein, “pharmaceutically acceptable excipient” includes solvents, dispersion media, coatings, antibacterial and antifungal agents, isotonic and absorption delaying agents, and the like that are physiologically compatible. Examples of pharmaceutically acceptable excipients include one or more of the following: water, saline, phosphate buffered saline, dextrose, glycerol, ethanol and the like, as well as combinations thereof. In some cases, the present composition includes isotonic agents, for example, sugars, polyalcohols such as mannitol, sorbitol, or sodium chloride. Pharmaceutically acceptable excipients may further include minor amounts of auxiliary substances such as wetting or emulsifying agents, preservatives or buffers, which enhance the shelf life or effectiveness of active agents, e.g., the ERα coligand, in the composition. The composition may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like. Suitable pharmaceutically acceptable excipients have been described in a variety of publications, including, for example, “Remington: The Science and Practice of Pharmacy”, 19th Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc.

The compositions of the present disclosures may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. In some cases, compositions are in the form of injectable or infusible solutions.

In some cases, the present pharmaceutical composition will be suitable for injection into a subject, e.g., will be sterile. For example, in some embodiments, the pharmaceutical composition will be suitable for injection into a subject, e.g., where the composition is sterile and is free of detectable pyrogens and/or other toxins. The composition can be formulated as a solution, microemulsion, dispersion, liposome, or other ordered structure suitable to high drug concentration. Sterile injectable solutions can be prepared by incorporating the active compound (i.e., an ERα coligand) in the required amount in an appropriate solvent with one or a combination of ingredients enumerated herein, as required, followed by sterile filtration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In some embodiments, an ERα coligand of the present disclosure is formulated in a sustained release dosage form that is designed to release the ERα coligand at a predetermined rate for a specific period of time. In certain embodiments, the active compound, e.g., the ERα coligand, may be prepared with a carrier that will protect the compound against rapid release, such as a controlled release formulation, including implants, transdermal patches, devices, e.g., ingestible devices, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art (see, e.g., Sustained and Controlled Release Drug Delivery Systems, J. R. Robinson. ed., Marcel Dekker, Inc., N.Y., 1978).

For oral preparations, an ERα coligand can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

In certain embodiments, an ERα coligand can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Unit dosage forms for oral administration such as syrups, elixirs, and suspensions may be provided wherein each dosage unit, for example, teaspoonful, tablespoonful, or tablet, contains a predetermined amount of the active agents, e.g., the ERα coligand, of the present disclosure. Similarly, unit dosage forms for injection or intravenous administration may include one or more agents that inhibit the activity of a neuronal activity-regulated protein in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of the active agent of the present disclosure calculated in an amount sufficient to produce the desired effect in association with a pharmaceutically acceptable diluent, carrier or vehicle. The specifications for the unit dosage forms of the present disclosure depend on the particular agent or agents employed and the effect to be achieved, and the pharmacodynamics associated with each agent in the subject.

Any of the therapeutic agents of the present disclosure may be formulated for use in any route of administration or dosage form disclosed herein.

An effective amount of the ERα coligand to achieve allosteric modulation of ER signaling, e.g., ER agonist-dependent, ERα-mediated signaling, may include a range of concentration of the ERα coligand at which the ERα coligand does not compete for binding with the ER agonist, e.g., estradiol, for the ligand binding site when the composition is administered to an individual.

In some cases, a composition of the present disclosure includes a pharmaceutically effective amount of an ERα coligand and a pharmaceutically effective amount of one or more ER agonists, i.e., ER agonists whose signaling mediated by ERα is allosterically and cell-type selectively modulated by the ERα coligand, in a pharmaceutically acceptable excipient. The ER agonists may be any suitable ER agonists. Suitable ER agonists include, but are not limited to, estrone (E1), 2-hydroxyestrone, 2-methoxyestrone, 4-hydroxyestrone, 15-alpha-hydroxyestrone, 16-alpha-hydroxyestrone, 16-beta-hydroxyestrone, 17β-estradiol (E2), 2-hydroxy-estradiol, 2-methoxy-estradiol, 4-hydroxy-estradiol, 16-oxoestradiol, estriol (E3), 16-epiestriol and 17-epiestriol, estetrol (E4), 27-hydroxycholesterol, dehydroepiandrosterone (DHEA), 7-oxo-DHEA, 7α-hydroxy-DHEA, 16α-hydroxy-DHEA, 7β-hydroxyepiandrosterone, Δ4-androstenedione, Δ5-androstenediol, 3α-androstanediol, 3β-androstanediol, 3-hydroxyestra-1,3,5,7-tetraen-17-one (equilin), 17α-dihydroequilin sulfate, 17α-estradiol sulfate, Δ^{8,9}-dehydroestrone sulfate, equilenin sulfate, 17β-dihydroequilin sulfate, 17α-dihydroequilenin sulfate, 17β-dihydroequilenin sulfate (0.5%) and salts or derivatives thereof. In some cases, a derivative of an ER agonist is a sulfate-conjugated derivative. In some cases, a composition of the present disclosure comprises synergistically effective amounts of: a) an ERα coligand of Formula I, Formula II, or Formula III; and b) an ER agonist. In some cases, a composition of the present disclosure comprises synergistically effective amounts of: a) 2′, 3′, 4′-THC; and b) one of the above-listed ER agonists.

In some cases, a composition of the present disclosure comprises synergistically effective amounts of: a) an ERα coligand of Formula I, Formula II, or Formula III; and b) a compound selected from E2, equilin, ethinylestradiol, estrone, estriol, 2-hydroxyestradiol, 4-hydroxyestradiol, 17α-estradiol, and 2-methoxyestradiol. In some cases, a composition of the present disclosure comprises synergistically effective amounts of: a) 2′, 3′, 4′-THC; and b) a compound selected from E2, equilin, ethinylestradiol, estrone, estriol, 2-hydroxyestradiol, 4-hydroxyestradiol, 17α-estradiol, and 2-methoxyestradiol.

The therapeutically effective amounts of the active agents in the present composition may be any suitable amounts and may vary depending on the intended effect of the ERα coligand and/or ER agonist when the composition is administered to an individual. Thus, in some case, a therapeutically effective amount of the ERα coligand may be an amount that is sufficient to treat a health condition or disease, or a symptom thereof, that is associated with ER signaling, e.g., a reduction in ER signaling due to menopause. A therapeutically effective amount of the ER agonist may be an amount that is sufficient to treat a health condition or disease, or a symptom thereof, that is associated with ER signaling, e.g., a reduction in ER signaling due to menopause. The therapeutically effective amount of the ERα coligand, when provided with the ER agonist(s) in the present composition, may vary, and may depend on the health condition or disease, or a symptom thereof, to be treated by administering the composition to the individual and on the ER agonist(s). In some cases, the therapeutically effective amount of the ERα coligand may be an amount that is sufficient to treat a side effect of administering the ER agonist(s).

The relative amount of the ERα coligand and ER agonist(s) in the present composition may be any suitable ratio, and may vary depending on the binding affinity to the respective ER binding sites of the active agents, pharmokinetic profiles of the active agents, the condition to be treated, the target tissue, etc. The ratio of the ERα coligand to an ER agonist may include a range of ratios at which the ERα coligand does not compete for binding with the ER agonist for the ligand binding site when the composition is administered to an individual. In some embodiments, the molar ratio of the ER agonist to the ERα coligand in the composition is 1:100 or less, e.g., 1:200 or less, 1:400 or less, 1:600 or less, 1:800 or less, including 1:1,000 or less, and is 1:10⁶ or greater, e.g., 1:10⁵ or greater, 1:10⁴ or greater, including 1:5,000 or greater. In some embodiments, the molar ratio of the ER agonist to the ERα coligand in the composition is in the range of 1:100 to 1:10⁶, e.g., 1:200 to 1:10⁵, 1:400 to 1:10⁴, including 1:600 to 1:5,000.

### Methods

### Methods of Treating

Also provided herein is a method of treating an individual for an ER-associated condition by administering a therapeutically effective amount of an ERα coligand, as described above, to an individual in need, thereby reprogramming ER agonist-dependent signaling in a tissue-selective manner The ER agonist-dependent signaling, e.g., ER agonist-dependent, ERα-mediated signaling, may be reprogrammed by the ERα coligand in any suitable tissue in any suitable manner, depending on the desired change in the ER agonist-dependent signaling at different tissues. In some cases, ER agonist-dependent signaling is enhanced or increased in a tissue-selective manner, and in some cases, ER agonist-dependent signaling is suppressed or reduced in a tissue-selective manner, as discussed above. In some cases, ER agonist-dependent signaling is ER agonist-dependent cell proliferation, transcriptional regulation, etc.

The ER agonist-dependent signaling, e.g., ER agonist-dependent, may be modulated by the ERα coligand in any suitable tissue-selective manner In some cases, the ERα coligand modulates ER agonist-dependent signaling tissue-selectively in breast tissue, uterus, bone, adipose tissue, brain tissue, ovaries, nerve tissue, kidney, heart, prostate, endothelial tissue, etc. Tissue-selective modulation may occur in healthy tissue, or pathological tissue, e.g., cancerous tissue. In some embodiments, administration of the ERα coligand reprograms ER agonist-dependent signaling such that modulation of ER agonist-dependent signaling is qualitatively or quantitatively different between bone and uterus; bone and breast tissue; bone and adipose tissue; adipose tissue and uterus; adipose tissue and breast tissue; brain and uterus; brain and breast tissue; and so forth. The reprogramming of ER agonist-dependent signaling may include modulation of any other ER agonist-dependent signaling that is qualitatively or quantitatively different between any other pairs of tissues.

The ER-associated condition may be any suitable health condition or disease that is caused by, exacerbated by, or otherwise associated with a change in ER agonist-dependent signaling, and that may be treated by administering the present ERα coligand to an individual suffering from the ER-associated condition. ER-associated conditions include, without limitation, breast cancer, endometrial cancer, colon cancer, pulmonary cancer, dementia, Alzheimer's disease, hot flashes, mood swings, insomnia, vaginal atrophy, vaginal dryness, dyspareunia, venous thromboembolism, gallbladder disease, obesity and diabetes.

In some cases the ER-associated condition includes symptoms of menopause, which may be caused by reduced ovary function due to age, disease (e.g., premature ovarian failure) and/or surgery (e.g., oophorectomy). Symptoms of menopause may include, without limitation, osteoporosis, hot flashes, vaginal atrophy, vaginal dryness, dyspareunia, obesity and/or diabetes. In some cases the ER-associated condition includes one or more side effects of administering exogenous ER agonist to an individual, e.g., side effects of menopausal hormone therapy to treat symptoms of menopause. Side effects of menopausal hormone therapy include, without limitation, cancer (e.g., breast cancer, endometrial cancer), venous thromboembolism and/or gallbladder disease, etc., that may be suitably treated by administering the present ERα coligand.

The individual to whom an ERα coligand or the present disclosure is administered may be any suitable individual. In some cases, the individual is an individual undergoing an age-induced menopause. In some cases, the individual is an individual approaching menopause (premenopause), where the circulating levels of reproductive hormones may be more variable and/or lower than the previously typical levels for the individual and/or symptoms of menopause are present in the individual. In some cases, the individual is perimenopausal, and may be within a 6- to 10-year phase that ends 12 months after the last menstrual period. In some cases, the individual is postmenopausal and has not experienced any menstrual flow for at least 12 months (without being pregnant or lactating).

In some embodiments, a method of the present disclosure includes co-administering one or more additional therapies with the ERα coligand to treat an individual for an ER-associated condition. The one or more additional therapies may be any suitable therapy, including, but not limited to, administration of menopausal hormone therapy, such as administration of estrogen with or without further co-administration of progesterone/progestins; and administration of other pharmaceutical agents, such as selective estrogen receptor modulators (SERMs), selective serotonin re-uptake inhibitors (SSRIs), and serotonin-norepinephrine reuptake inhibitors (SNRIs). Any suitable menopausal hormone therapy may be co-administered to the individual, as described in, e.g., Santoro et al., Glob. libr. women's med., (2012) DOI 10.3843/GLOWM.10083, which is incorporated herein by reference. Suitable estrogen for co-administering with the ERα coligand include, without limitation, piperazine estrogen sulfate; micronized estradiol; conjugated estrogens; ethinyl estradiol; estradiol valerate; esterified estrogens; and estriol. A progestin may include any suitable synthetic progestogen that mimics the physiological function of progesterone. A progestin includes, without limitation, retroporgesterone, 17α-hydroxyprogesterone, haloprogesterone, medrogestone, proligestone, 19-norprogesterone, 17α-ethynyltestosterone, 19-nortestosterone, 17α-spirolactone, and derivatives thereof.

In some cases, a method of the present disclosure comprises administering synergistically effective amounts of: a) an ERα coligand of Formula I, Formula II, or Formula III; and b) an ER agonist. In some cases, a method of the present disclosure comprises administering synergistically effective amounts of: a) 2′,3′,4′-THC; and b) an ER agonist. In some cases, a method of the present disclosure comprises administering synergistically effective amounts of: a) an ERα coligand of Formula I, Formula II, or Formula III; and b) a compound selected from E2, equilin, ethinylestradiol, estrone, estriol, 2-hydroxyestradiol, 4-hydroxyestradiol, 17α-estradiol, and 2-methoxyestradiol. In some cases, a method of the present disclosure comprises administering synergistically effective amounts of: a) 2′, 3′, 4′-THC; and b) a compound selected from E2, equilin, ethinylestradiol, estrone, estriol, 2-hydroxyestradiol, 4-hydroxyestradiol, 17α-estradiol, and 2-methoxyestradiol.

Co-administering the additional therapies may include any suitable means of co-administering, depending on the additional therapy. In some cases, the additional therapy is administered concurrently with the ERα coligand. In some cases, where the additional therapy is an additional pharmaceutical agent, the additional pharmaceutical agent is administered in the same composition as a composition containing the ERα coligand. In some embodiments, the additional therapy is administered sequentially with the ERα coligand.

The ERα coligand of the present disclosure may be administered by any suitable means, including parenteral, non-parenteral, subcutaneous, topical, intraperitoneal, intrapulmonary, intranasal, and intralesional administration (e.g., for local treatment). Administration may be systemic or local. Parenteral infusions include, but are not limited to, intramuscular, intravenous, intra-arterial, intraperitoneal, or subcutaneous administration. In addition, the ERα coligand is suitably administered by pulse infusion, particularly with declining doses. In some cases, the dosing is given by injections, such as intravenous or subcutaneous injections, depending in part on whether the administration is brief or chronic. In some instances, therapeutic agents of the present disclosure are administered orally, such as through the digestive tract (enteral administration), buccal, sublabial, or sublingual administration. Such dosage forms may be pills, tablets, capsules, time-release formulations, osmotic controlled release formulations, solutions, softgels, hydrogels, suspensions, emulsions, syrups, orally disintegrating tablets, films, lozenges, chewing gums, mouthwashes, ointments, and the like.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is advantageous to formulate parenteral compositions in dosage unit form for ease of administration and uniformity of dosage. The dosages of the ERα coligand of the present disclosure are generally dependent on (a) the unique characteristics of the active compound and the particular therapeutic or prophylactic effect to be achieved, and (b) the limitations inherent in the art of compounding such an active compound for the treatment of individuals.

An exemplary, non-limiting range for a therapeutically or prophylactically effective amount of an ERα coligand is 0.1-20 mg/kg, e.g., from 1 mg/kg to 10 mg/kg. In some embodiments, the dosage is from 50-600 mg/m² (e.g. 375 mg/m²). It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the present invention.

The dosage administered will, of course, vary depending upon known factors such as the pharmacodynamic characteristics of the particular agent, its mode and route of administration, the age, health, and weight of the recipient, the nature and extent of symptoms, the kind of concurrent therapy, the frequency of administration, and the effect desired. For example, a daily dosage of active ingredient can be about 0.01 to 100 milligrams per kilogram of body weight. In some cases, 1 to 5, including 1 to 10 milligrams per kilogram per day given in divided doses 1 to 6 times a day or in sustained release form, may be effective to obtain desired results.

In some embodiments, the administering includes administering an amount of the ERα coligand sufficient to achieve a local concentration in a target tissue that is 1/10 or less, e.g., 1/20 or less, 1/50 or less, 1/100 or less, including 1/1,000 or less, and a local concentration in a target tissue that is 1/100,000 or more, e.g., 1/50,000 or more, 1/20,000 or more, 1/10,000 or more, including 1/5,000 or more of the in vitro IC₅₀ at which the coligand competes with estrogen for binding to the estrogen binding site of the ER. In some embodiments, the amount of the ERα coligand administered is sufficient to achieve a local concentration in a target tissue that is in the range of 1/100,000 to 1/10, e.g., 1/50,000 to 1/20, 1/20,000 to 1/50, 1/10,000 to 1/100, including 1/10,000 to 1/1,000 of the in vitro IC₅₀ at which the coligand competes with estrogen for binding to the estrogen binding site of the ER.

Administering the ERα coligand may suppress or reduce ER signaling, e.g., ER agonist-dependent, ERα-mediated signaling, in a tissue-selective manner, compared to ER signaling, e.g., ER agonist-dependent ERα-mediated signaling, in the absence of the ERα coligand, by 10% or more, e.g., 20% or more, 30% or more, 40% or more, 50% or more, 60% or more, 70% or more, 80% or more, 90% or more, and up to about 100%, and in some embodiments, by 100% or less, e.g., 95% or less, 90% or less, 85% or less, 80% or less, 70% or less, 60% or less, 50% or less, including 40% or less. In some cases, administering the ERα coligand suppresses or reduces ER signaling, e.g., ER agonist-dependent, ERα-mediated signaling, in a tissue-selective manner, compared to ER signaling, e.g., ER agonist-dependent ERα-mediated signaling, in the absence of the ERα coligand, by 10 to 100%, e.g., 20 to 100%, 30 to 95%, 40 to 95%, including 50 to 90%.

Administering the ERα coligand may enhance or increase ER signaling, e.g., ER agonist-dependent, ERα-mediated signaling, in a tissue-selective manner, compared to ER signaling, e.g., ER agonist-dependent ERα-mediated signaling, in the absence of the ERα coligand, by 1.5 fold or more, e.g., 2 fold or more, 2.5 fold or more, 3 fold or more, 4 fold or more, 5 fold or more, 10 fold or more, including 15 fold or more, and in some cases, by 100 fold or less, e.g., 50 fold or less, 30 fold or less, 20 fold or less, 10 fold or less, including 3 fold or less. In some embodiments, administering the ERα coligand enhances or increases ER signaling, e.g., ER agonist-dependent, ERα-mediated signaling, in a tissue-selective manner, compared to ER signaling, e.g., ER agonist-dependent ERα-mediated signaling, in the absence of the ERα coligand, by 1.5 to 100 fold, e.g., 2 to 50 fold, 2 to 30 fold, 2 to 20 fold, including 2 to 10 fold.

### Methods of Identifying an ERα Coligand

Also provided herein is a method of identifying an active agent, e.g., a compound, that allosterically modulates ERα signaling in a cell-type selective manner The method may include contacting a first cell type functionally expressing ERα, or ERα and ERβ, with a candidate agent; contacting a second cell type functionally expressing ERα, or ERα and ERβ, with the candidate agent; measuring a first and second modulatory effects of the candidate agent on ER agonist-dependent ERα signaling in cells of the first and second cell types, respectively, wherein the ERα coligand does not compete with an ER agonist for binding to ERα, and wherein the candidate agent is determined to be an active agent that allosterically modulates ERα signaling in a cell-type selective manner if the first modulatory effect and second modulatory effect are quantitatively or qualitatively different.

The present method may be used to assay any suitable candidate agent, including, but not limited to, small molecules, polypeptides, etc. The modulatory effect on ER agonist-dependent ERα signaling may be measured using any suitable parameter, including, but not limited to, cell proliferation, reporter expression, ER localization to DNA, etc. The modulatory effect in the first cell type may be measured based on a parameter of ER agonist-dependent ERα signaling that is the same or different from the parameter of ER agonist-dependent ERα signaling measured in the second cell type. The modulatory effect on the first cell type may be different from the modulatory effect on the second cell type by a difference in magnitude, a difference in sign, and/or a difference in the lack or presence of a modulatory effect.

### Utility

An ERα coligand of the present disclosure finds use in a variety of therapeutic applications, where it is desirable to modulate ER agonist-dependent ERα signaling in a cell type-selective manner The ERα coligand may selectively suppress ERα-mediated signaling in cell types where ERα-mediated signaling results in excess proliferation of cells and increased risk of cancer, while preventing loss of cells due to ERα-mediated signaling in other cell types. Thus, the ERα coligand may suppress side effects of hormonal therapies that include administration of an ERα agonist, such as menopausal hormone therapy. Side effects may include side effects of administering an estrogen, e.g., estradiol, and analogs and derivative thereof; and side effects of co-administering a progestin/progesterone with the estrogen.

In some cases, use of the ERα coligand can prolong the duration of hormonal therapy that include administration of an ERα agonist, such as menopausal hormone therapy, without substantially or significantly increasing the risk of developing side effects of the hormonal therapy, such as the risk of developing cancer. In some cases, the use of the ERα coligand may allow menopausal hormone therapy without co-administration of progestin/progesterone.

In some cases, the ERα coligand may be administered to an individual to potentiate ER agonist-dependent ERα signaling where the endogenous level of circulating estrogen is low, e.g., after menopause. In certain embodiments, the ERα coligand may be used to reduce the risk or slow progression of osteoporosis due to reduced circulating estrogen levels after menopause.

In some embodiments, the ERα coligand may be administered to an individual to potentiate ER agonist-dependent ERβ signaling, e.g., to promote the antiproliferative actions of ERβ, and thereby to reduce the risk of an individual developing an ER-associated condition, e.g., cancer.

### EXAMPLES

The following examples are put forth so as to provide those of ordinary skill in the art with a complete disclosure and description of how to make and use the disclosed subject matter, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Materials and Methods

The following materials and methods were used in the Examples.

### Compounds

Compounds 2′, 3′, 4′-trihydroxychalcone (2′, 3′, 4′-THC) and 2, 2′, 4′-trihydroxychalcone (2, 2′, 4′-THC) were obtained from INDOFINE Chemical Company (Hillsborough, N.J.). All other compounds were obtained from Sigma Aldrich (St. Louis, Mo.). The chemicals were dissolved in ethanol and used at a final concentration of 0.1%.

### Cell Culture

Human osteosarcoma cell lines expressing a tetracycline-regulated ERα (U205-ERα ) and ERβ (U205-ERβ) cDNA were prepared, characterized, and maintained as previously described (Kian Tee, M., et al. Estradiol and selective estrogen receptor modulators differentially regulate target genes with estrogen receptors alpha and beta. Mol Biol Cell 15, 1262-1272 (2004)). Cells were maintained in Dulbecco's Modified Eagle Medium (DMEM)/F-12 supplemented with 5% stripped fetal bovine serum (Gemini Bio-Products, Sacramento, Calif.), 100 units/mL of penicillin and streptomycin, 50 μg/mL fungizone® and 2 mM glutamine. All cells were continuously maintained in phenol red-free media supplemented with 50 μg/mL of hygromycin B and 500 μg/mL of zeocin. MCF-7 breast cancer cell lines were obtained from American Type Culture Collection (ATCC) and maintained in DMEM/F-12 supplemented with 10% fetal bovine serum (Gemini Bio-Products), 100 units/mL of penicillin and streptomycin, 50 μg/mL fungizone and 2 mM glutamine. All cells were continuously maintained in phenol red-free media.

### Transfection and Luciferase Assays

U2OS cells were transfected with 3 μg of a plasmid containing the ERE upstream of the minimal thymidine kinase luciferase promoter and 1 μg of cytomegalovirus (CMV)-ERα or CMV-ERβ by electroporation. Cells were treated for 24 hours then lysed and assayed for luciferase activity according to the manufacturer's protocol (Promega Corp., Madison, Wis.) using a luminometer.

### Competitive Estrogen Receptor Binding Assays

U205-ERα cells grown in 12-well dishes were treated for 24 hours with and without 1 μg/ml doxycycline. After the treatment, cells were incubated at 37° C. for 1 h with 5 nM [³H]-E2 (specific activity 87.6 Ci/mmol; PerkinElmer Life Science, Waltham, Mass.) in the presence of increasing concentrations of 2′, 3′, 4′-THC. After washing the cells with 0.1% bovine serum albumin in phosphate buffered saline (PBS), 100% ethanol (ETOH) was added. Radioactivity was measure in the samples with a scintillation counter. Specific binding of [³H]-E2 was calculated as the difference between total and nonspecific binding in counts per minute (CPM).

### RNA Extraction and Quantitative Real-Time PCR

Total RNA was extracted and then treated with DNAse using the Aurum™ Total RNA Mini Kit (Bio-Rad Laboratories, Hercules, Calif.). Reverse transcription reactions were performed using the iScript™ cDNA Synthesis Kit with 1 μg of total RNA according to manufacturer's protocol. Quantitative polymerase chain reaction (qPCR) z0 was performed with a Bio-Rad CFX96™ Thermal Cycler System using SsoFast™ Eva Green® Supermix (Bio-Rad). Mean±SEM was calculated using Prism curve-fitting program (GraphPad Software, Inc., La Jolla, Calif.).

### Microarray and Data Analysis

Total cellular RNA was isolated utilizing the Aurum™ RNA isolation kit (Bio-Rad, Hercules, Calif.) per the manufacturer's directions. RNA isolates were first quantified by nanodrop®, and then qualitatively evaluated by the Bio-Rad Experion™ system per the manufacturer's instructions. Biotin-labeled cRNA samples were prepared using 750 ng of total RNA. Biotin-labeled samples were evaluated by both 260/280 absorbance spectrophotometry and capillary electrophoresis. Labeled cRNA samples were hybridized overnight against Human genome HG U133A-2.0 GeneChip® arrays, (Affymetrix, Santa Clara, Calif.). All treatments were done in triplicate with the same batch of microarrays. The data was analyzed as previously described (Saunier, E. F., et al. Estrogenic plant extracts reverse weight gain and fat accumulation without causing mammary gland or uterine proliferation. PloS one 6, e28333 (2011)).

### Chromatin Immunoprecipitation (ChIP)

U2OS-ERα cells were plated at 80% confluency and treated for 24 hours with 1 μg/μl doxycycline followed by treatment with vehicle, 10 nM E2, 5 μM 2′, 3′, 4′-THC or the combination of E2 and 2′, 3′, 4′-THC for 1 or 2 hours. Cells were fixed with 11X formaldehyde solution for 10 minutes at 37° C. and the reaction was quenched for 2 minutes with 1.25M glycine solution as previously described (Cvoro, A., et al. Distinct Roles of Unliganded and Liganded Estrogen Receptors in Transcriptional Repression. Mol Cell 21, 555-564 (2006)). Cells were washed and collected in collecting buffer (100 mM Tris-HCl pH 9.4 and 10 mM dithiothreitol (DTT)) supplemented with a protease inhibitor cocktail. Pellets were frozen overnight at −80° C. then were thawed on ice and lysed (50 mM Tris pH 7.4, 150 mM NaCl, 10 mM ethylenediaminetetraacetic acid (EDTA), 0.5 mM ethylene glycol tetraacetic acid (EGTA), 0.5% Triton™ X-100 and 1X protease inhibitor cocktail). Cell lysates were centrifuged and cell pellet was resuspended in RIPA buffer. Samples were sonicated and supernatant was collected. Approximately 5% of each sample was taken for input and stored at 4° C. Immune complexes were collected using magnetic sepharose beads (GE Healthcare, Pittsburgh, Pa.) equilibrated in radioimmunoprecipitation assay (RIPA) buffer. Complexes were collected over a period of 3 to 4 hours rotating at 4° C. After complexes were collected DNA was eluted overnight with elution solution (1% sodium dodecyl sulfate (SDS), 0.1M NaHCO₃) at 65° C. Eluted DNA was cleaned and concentrated using the ChIP DNA Clean and Concentrator™ (Zymo Research, Irvine, Calif.). Anti-ERα antibody (HC-20), normal mouse IgG (sc-2025, Santa Cruz Biotechnology, Santa Cruz, Calif. or anti-SRC-2 (ab-9261, NCOA2, Abcam, Cambridge, Mass.) were used for immunoprecipitation.

### Cell Proliferation Studies

MCF-7 cells were plated at a density of 50,000 cells per well in 6-well tissue culture plates in DMEM/ F-12 supplemented with 5% stripped fetal bovine serum (FBS). At following day, the cells were treated with vehicle, 1 nM E2 in the absence and presence of increasing doses of 2′, 3′, 4′-THC (1, 5 and 10 μM) for 5 days. The cells were then detached with trypsin, neutralized with media containing serum and well suspended by pipetting and vortex. The cell suspensions were then counted using a Coulter Counter® (Beckman)

### Flow Cytometry

MCF-7 cells were plated at a density of 500,000 cells per well in 6-well tissue culture dishes in DMEM/ F-12 supplemented with 5% stripped FBS for 24 hours and then switched to serum-free media for 24 hours. The cells were then treated with vehicle, 0.1 nM E2 in the absence and presence of increasing doses of 2′, 3′, 4′-THC (1, 5 and 10 μM) for 24 hours. The cells were then washed with PBS, detached with trypsin and collected by centrifugation. The cell pellets were washed with cold PBS followed by centrifuge at room temperature and re-suspended in 500 μl PBS containing 5 μg/μl propidium iodide, 0.1% of Triton™ X-100, 0.1% of sodium citrate, and 10 μg/ml of RNase. Samples were then run on Cytomics™ FC-500 (Beckman Coulter) in the cell cytometry facility at UC Berkeley and data was analyzed using FlowJo 7.6.5.

### In Silico Modeling

The structure of the ERα ligand binding domain (LBD) was obtained from the Protein Data Bank (PDB) with accession number lERE. An example of an amino acid sequence of an ERα LBD is as follows:

(SEQ ID NO: 1)
SKKNSLALSL TADQMVSALL DAEPPILYSE YDPTRPFSEA
SMMGLLTNLA DRELVHMINW AKRVPGFVDL TLHDQVHLLE
CAWLEILMIG LVWRSMEHPG KLLFAPNLLL DRNQGKCVEG
MVEIFDMLLA TSSRFRMMNL QGEEFVCLKS IILLNSGVYT
FLSSTLKSLE EKDHIHRVLD KITDTLIHLM AKAGLTLQQQ
HQRLAQLLLI LSHIRHMSNK GMEHLYSMKC KNVVPLYDLL
LEMLDAHRLH APT

The PRODRG server was used to produce the topology files for modeling the E2 and 2′, 3′, 4′-THC structure. The receptor (1ERE) and ligand (E2 and 2′, 3′, 4′-THC) structures were subsequently loaded into the Hex Protein Docking program. Prior to docking the structures, all water molecules and hetero molecules were manually removed by editing each PDB file. Shape and electrostatics were used as restrictive parameters to model binding between the receptor and ligand. Modeling results were visualized using PyMol (The PyMOL Molecular Graphics System, Version 1.7.4 Schrodinger, LLC). The program LigPlot was then used to generate schematic diagrams that illustrate the pattern of interactions between the 3-D coordinates of the protein and bound ligand.

### Protein Thermostability

Purified ERα LBD containing amino acids 315-545 used by Brzozowski et al (Brzozowski, A. M., et al. Molecular basis of agonism and antagonism in the oestrogen receptor. Nature 389, 753-758 (1997)) for crystallization with a histidine-tag that was custom made (GenScript). All reactions were set up in 20 μL reactions in 96 well plates with purified ERα LBD at a concentration of 0.6 μg/μL and 10x SPYRO® orange dye (Invitrogen). The ERα LBD was incubated with a concentration of 5 μM 2′, 3′, 4′-THC and increasing doses of E2 (1 nM, 10 nM, 100 nM) and compared to E2 doses alone. Thermal melting experiments were performed on the Viia™7 instrument (Life Technologies) melt curve program with a ramp rate of 0.05° C./s, and the ramp goes to 95° C. Melting temperatures were analyzed with Protein Thermal Shift Software (Life Technologies) to identify the midpoint of the transition with an analysis of the first derivative. All experiments were performed with triplicate samples.

### Animal Studies

8 week old C57b1/6J female ovariectomized or intact mice were purchased from Jackson Laboratories, Sacramento, Calif. Mice were housed and maintained according to OLAC standard procedures in the NAF facility at UC Berkeley, Calif. All mice were fed a soy-free chow diet 2916.15 (Harlan Laboratories, Livermore, Calif.) starting one week before osmotic pump implantation or 2′, 3′, 4′-THC injection. Pumps Model 2006 were purchased from Alzet and filled with vehicle, 1 μg 1713-estradiol, 2 mg of 2′, 3′, 4′-THC or the combination of 2′, 3′, 4′-THC and estradiol. All drugs were dissolved in sterile vehicle consisting of 50% dimethylsulfoxide (DMSO), 25% ETOH and 25% deionized water. Pumps were handled with sterile gloves and filled using a 27 gauge filling tube and 1 mL syringe. All pumps were placed in 1X PBS in 15 mL sterile conical tubes and incubated overnight at 37° . Pumps were surgically implanted into 8 week old C57b1/6J female ovariectomized mice (Jackson Laboratory, Sacramento, Calif.) posterior to the scapula and left for a duration of 4 weeks. Intact mice were injected intraperitoneal with 2′, 3′, 4′-THC every day for 3 weeks. Mice were weighed once a week for the duration of the experiment. The intraperitoneal gonadal fat was removed and weighed. Uterine tissue was collected, fluid drained and then weighed.

### RNA Extraction and Real-Time PCR of Animal Tissues

Tissues were dissected and immediately frozen in liquid nitrogen. Before RNA isolation, tissues were homogenized in PureZOL™ using the MP FastPrep™-24 for 40 seconds. Total RNA was extracted and then treated with DNAse using the Aurum™ Total RNA Mini Kit for Fatty and Fibrous Tissue (Bio-Rad Laboratories, Hercules, Calif.). Reverse transcription reactions were performed using the iScript™ cDNA Synthesis Kit with 1 μg of total RNA according to manufacturer's protocol. qPCR was performed with a Bio-Rad CFX96™ Thermal Cycler System using SsoFast™ Eva Green® Supermix (Bio-Rad). Mean±SEM was calculated using Prism curve-fitting program (GraphPad Software, Inc., San Diego, Calif.).

Uterine Tissue Slide Preparation Uterine tissue was removed and trimmed of excess adipose tissue. Tissues were fixed in formalin for 24 hours then transferred to 50% ETOH for 1 hour followed by 70% ethanol (ETOH) for another hour. After fixation tissues were sent to Histopathology Reference Laboratory, Hercules Calif. where they were paraffin embedded, sectioned and stained with hematoxylin and eosin for morphological exam

### Example 2

### 2′, 3′, 4′-THC Acts an ER Coagonist in U2OS Bone Cells

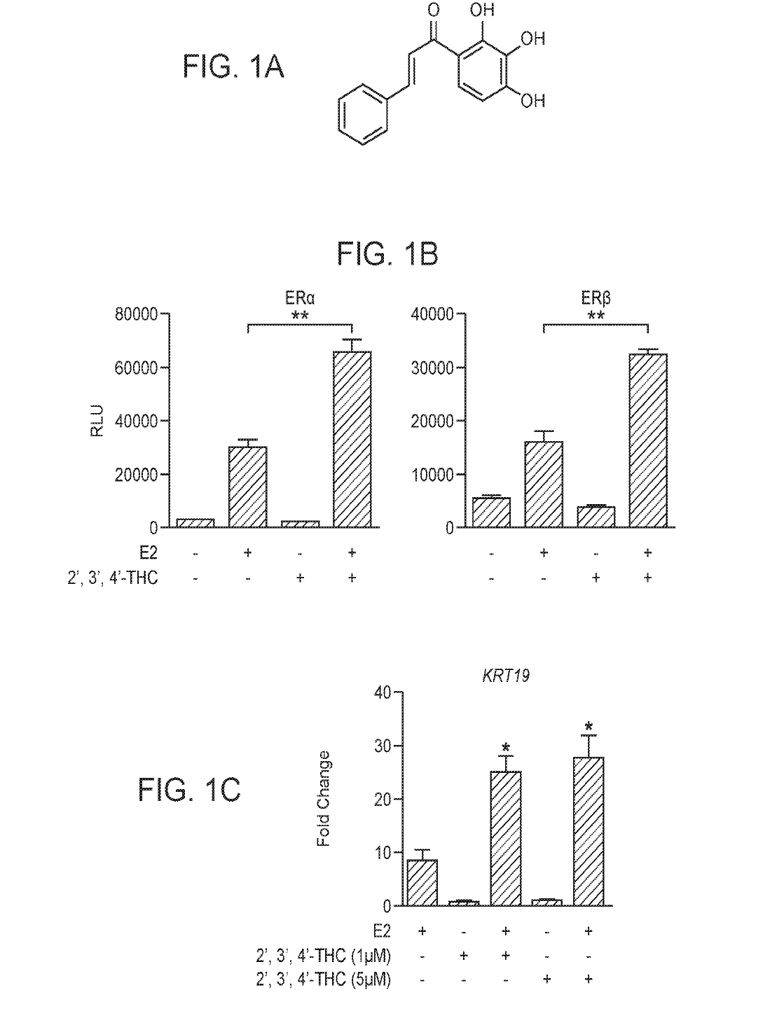

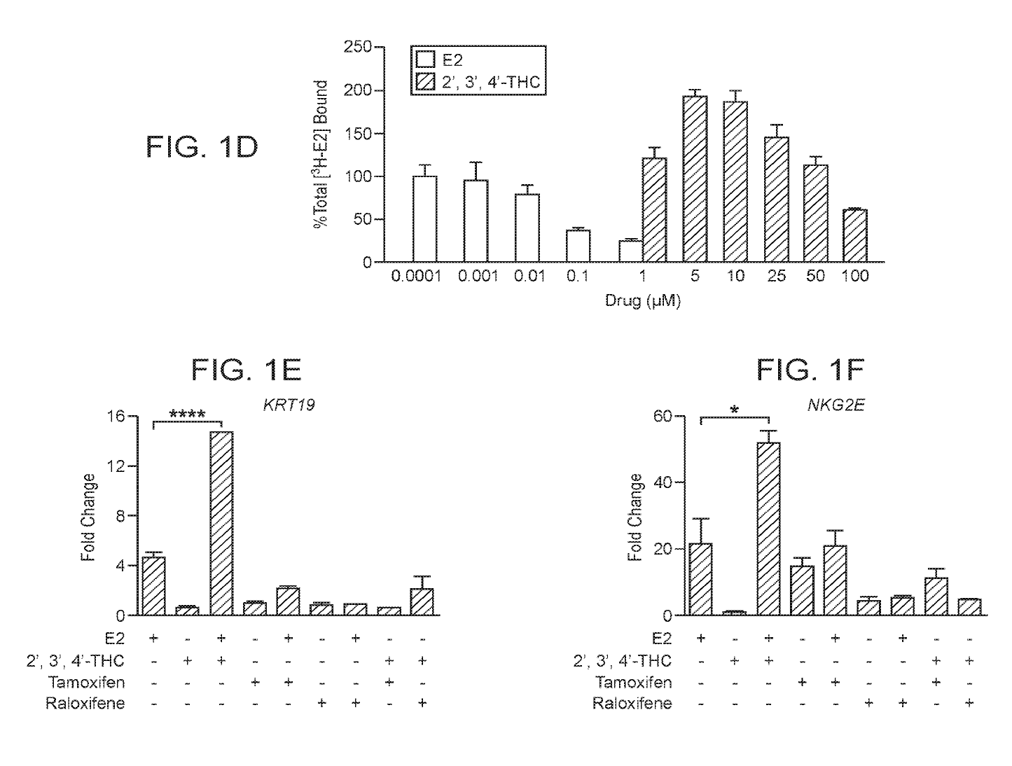

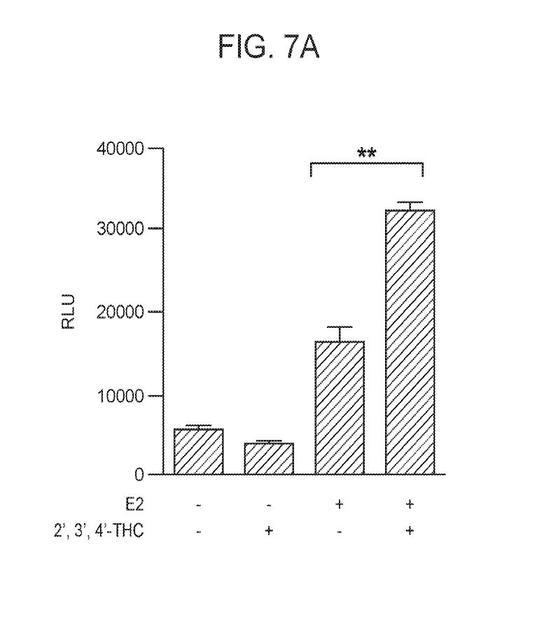

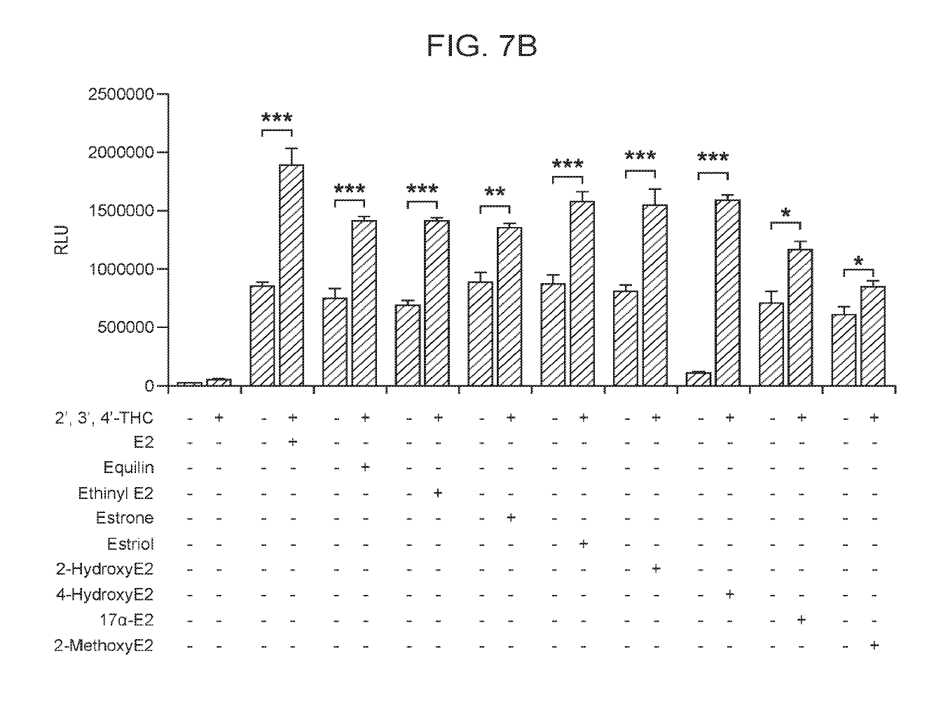

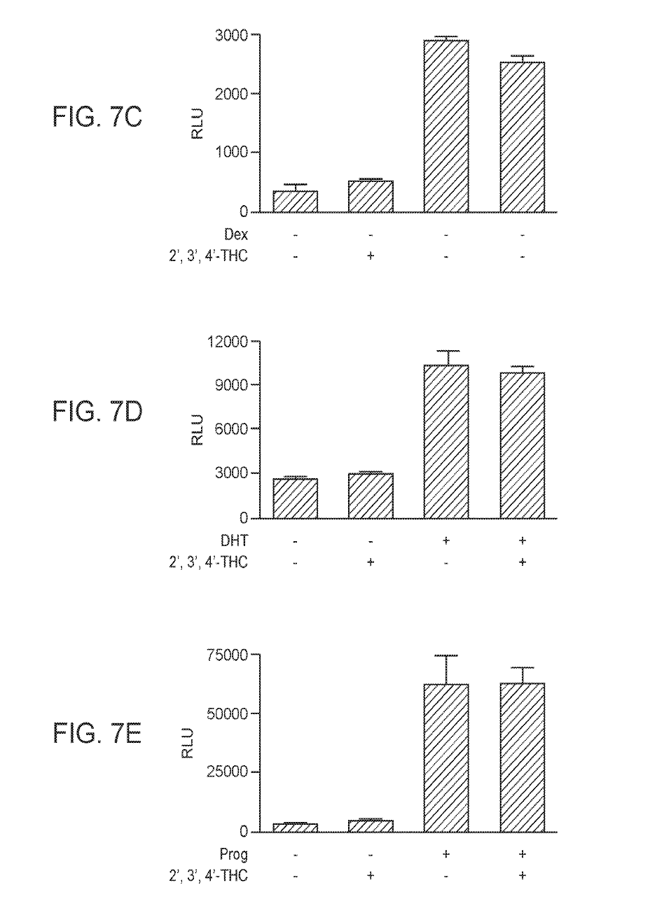

The flavanone liquiritigenin was previously isolated from the plant Glycyrrhizae uralensis Fisch and was shown to be an ERβ-selective agonist. To identify compounds that alter the activity of E2 by possibly acting as a coligand, compounds with a similar molecular structure and weight as liquiritigenin were screened for their ability to synergize with E2 in transfection assays. 2′, 3′, 4′-THC, a chalcone (FIG. 1a) which has the same molecular weight as liquiritigenin was selected for further studies based on the initial screening. 2′, 3′, 4′-THC was inactive on an ERE upstream of the luciferase gene in U2OS cells transfected with ERα or ERβ, whereas it produced a synergistic increase in luciferase activity with E2 in the presence of both ERα (FIG. 1b) or ERβ (FIG. 7a). Because ERα is the major receptor in bone and adipose tissue and the activation of ERα is associated with increased cancer risk by estrogens, the effects of 2′, 3′, 4′-THC on ERα actions were studied. To explore potential synergy between 2′, 3′, 4′-THC and E2, their effects were examined on the known ERα target gene, keratin 19 (KRT19). A synergistic activation of the KRT19 gene was observed with 1 and 5 μM 2′, 3′, 4′-THC in the presence of E2 (FIG. 1c). Because the synergy was greater with 5 μM this concentration was used in subsequent studies. Competition binding studies were performed to determine if 2′, 3′, 4′-THC competes with [³H]-E2 binding in U205-ERα cells. Surprisingly, 5 or 10 μM 2′, 3′, 4′-THC produced an approximate 2-fold increased [³H]-E2 binding in U205-ERα cells (FIG. 1d). 2′, 3′, 4′-THC did not compete for [³H]-E2 binding in U205-ERα cells until 100 μM (FIG. 1d), which was a 100-fold higher than required for synergy (FIG. 1c). The IC₅₀ for 2′, 3′, 4′-THC was approximately 10,000-fold higher than E2 in U205-ERα cells and with purified ERα assayed by fluorescent polarization. These studies demonstrate that 2′, 3′, 4′-THC synergistic activation exceeds E2 maximum dose for E2 binding to ERα by 100 fold.

No activation of NKG2E-TK-Luc occurred with 2′, 3′, 4′-THC alone, but it produced a synergistic response at 1 μM and a maximal response at 5 μM. A similar synergistic action of 2′, 3′, 4′-THC on NKG2E-TK-Luc was observed with other estrogens, including equilin, ethinyl estradiol, estrone and estriol and the E2 metabolites, 2-hydroxyestradiol and 4-hydroxyestradiol (FIG. 7b). A smaller synergistic activation was observed with 17α-estradiol and 2-methoxyestradiol. The synergy was specific for ERs, because no synergy occurred with the human glucocorticoid (FIG. 7c), androgen (FIG. 7d) and progesterone-B (FIG. 7e) receptors.

FIGS. 1a-1f2′, 3′, 4′-THC synergizes the E2 induced transcriptional activity of ERE-tk luciferase in U2OS cells expressing ERα or ERβ. (FIG. 1a) Chemical structure of 2′, 3′, 4′-THC. (FIG. 1b) U2OS cells cotransfected with ERE tk-luciferase and ERα (left panel) or ERβ (right panel). After transfection the cells were treated for 24 hours with 10 nM estradiol and 5 μM 2′, 3′, 4′-THC alone or in combination. Cells were lysed and luciferase activity was measured. RLU is relative light units. Error bars are the Mean±SEM. **P≤0.01. (FIG. 1c) U2OS-ERα cells were treated with 1 or 5 μM E2 in the absence or presence of 5 μM 2′, 3′, 4′-THC for 24 h. The total cellular RNA was extracted and real-time PCR for KRT19 mRNA was performed as described in Materials and Methods. (FIG. 1d) Competitive binding curve of E2 and 2′, 3′, 4′-THC in U2OS-ERα cells. Cells were treated with 5 nM [³H]-E2 and increasing doses of E2 or 2′, 3′, 4′-THC for 1 hour at 37° . Total [³H]-E2 binding was measured with a scintillation counter (FIG. 1e) Gene expression of KRT19 and (FIG. 1f) NKG2E in U2OS-ERα cells treated with 10 nM estradiol, 5 μM 2′, 3′, 4′-THC, 1 μM tamoxifen or raloxifene alone or in combination for 24 hours. Error bars are ±SEM. *P≤0.05, **** P≤0.0001.

FIG. 7a-7e. The synergistic stimulation of E2 activation of reporter genes is specific for ER. U2OS cells cotransfected with (A) ERE-TK-Luc and ERI3 and treated with 10 nM of E2 and 5 μM of 2′, 3′, 4′-THC alone or in combination for 24 hours. (B) U2OS cells cotransfected with NKG2E-TK-Luc and ERα and treated with 10 nM of E2, 10 nM equilin, 10 nM ethinyl E2, 100 nM estrone, 100 nM estriol, 100 nM 2-hydroxyE2, 100 nM 4-hydroxyE2, 100 nM 17α-E2 and 100 nM 2-methoxyE2 and 5 μM of 2′, 3′, 4′-THC alone or in combination for 24 hours. (C) U2OS cells cotransfected with GRE-TK-Luc and human (h) GR and treated with 10 nM of dexamethasone (Dex) and 5 μM of 2′, 3′, 4′-THC alone or in combination for 24 hours. (D) U2OS cells cotransfected with human tyrosine aminotransferase (TATS)-luciferase reporter gene and hAR and treated with 10 nM of dihydrotestosterone (DHT) and 5 μM of 2′, 3′, 4′-THC alone or in combination for 24 hours. (E) U2OS cells cotransfected with TAT3-luciferase reporter gene and hPR-B and treated with 10 nM of progesterone (Prog) and 5 μM of 2′, 3′, 4′-THC alone or in combination for 24 hours. After transfection, luciferase activities were measured as described in the Methods. RLU is relative light units and the data shown are mean±SEM.

### Example 3

### 2′, 3′, 4′-THC Behaves as a Coagonist, Whereas Selective Estrogen Receptor Modulators (SERMs) Function as Antagonists on E2-Mediated Gene Regulation in U2OS-ERα Cells

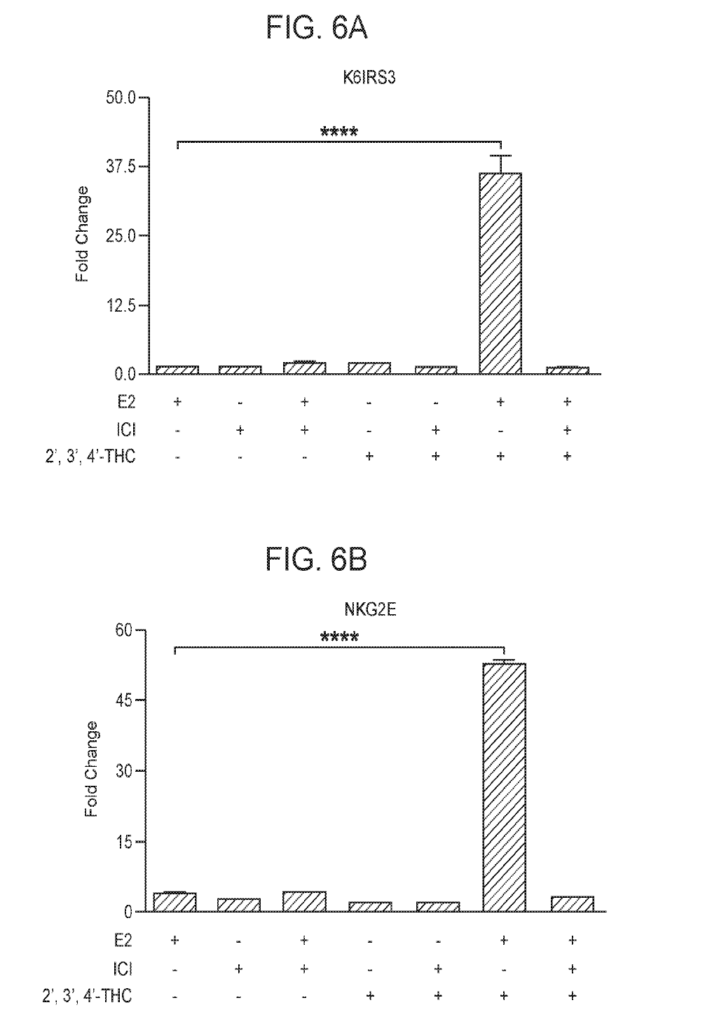

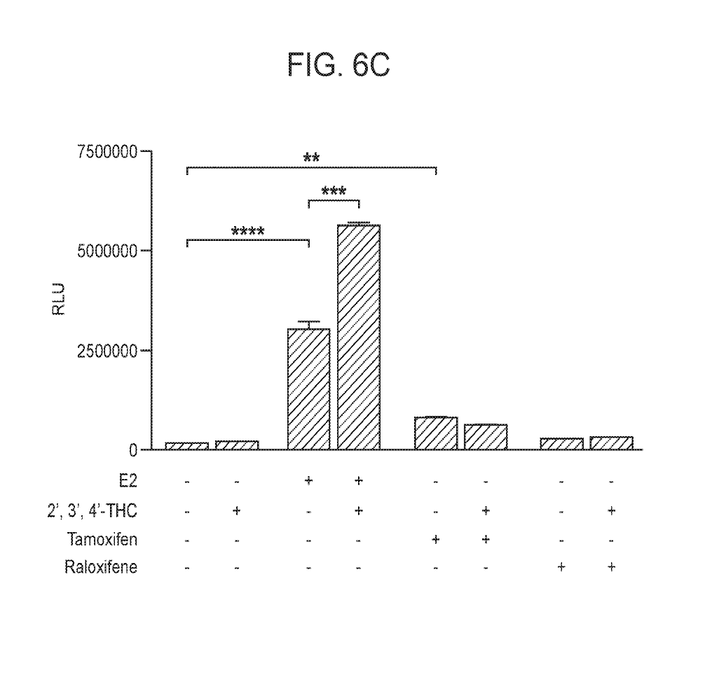

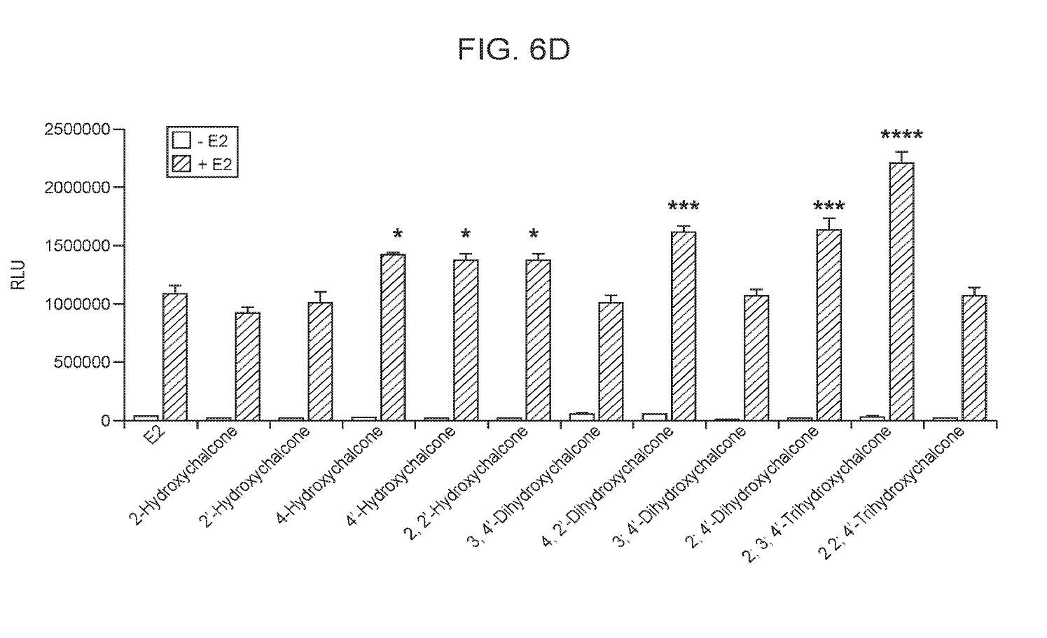

To compare the activity of 2′, 3′, 4′-THC to SERMs on endogenous gene regulation, their effects on the expression of the KRT19 and NKG2E genes were examined E2 induced KRT19 gene expression in U2OS-ERα cells, whereas no effect was observed with 2′, 3′, 4′-THC, tamoxifen or raloxifene (FIG. le). The 2′, 3′, 4′-THC/E2 combination produced a synergistic activation of KRT19, whereas tamoxifen and raloxifene blocked E2-induced expression (FIG. 1e). E2, tamoxifen and raloxifene activated NKG2E gene expression whereas no effect was observed with 2′, 3′, 4′-THC (FIG. 1f). A synergistic activation of the NKG2E gene occurred with the 2′, 3′, 4′-THC/E2 combination, whereas raloxifene antagonized the E2 effect. No synergy was observed with 2′, 3′, 4′-THC in combination with tamoxifen or raloxifene. Similar results were observed in transfection assays. NKG2E-TK-Luc was activated by E2, tamoxifen and raloxifene, but 2′, 3′, 4′-THC produced a synergistic activation only with E2 (FIG. 6c). These data demonstrate that 2′, 3′, 4′-THC does not behave like classical SERMs in U2OS cells, which antagonize E2-induced gene activation. In contrast, 2′, 3′, 4′-THC acts as a coagonist by potentiating the effects of E2. A structurally similar chalcone, 2, 2′, 4′-THC did not produce the synergistic activation of ERE-TK-Luc observed with 2′, 3′, 4′-THC (FIG. 6d).

To determine if 2′, 3′, 4′-THC is a coagonist of E2 on genes other than KRT19 and

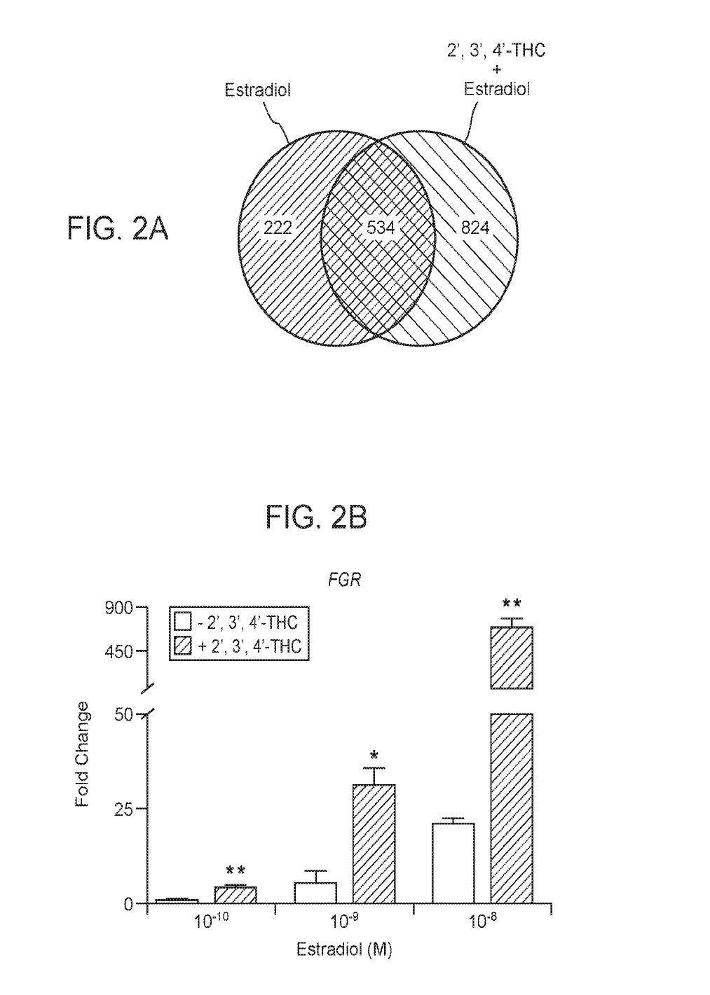

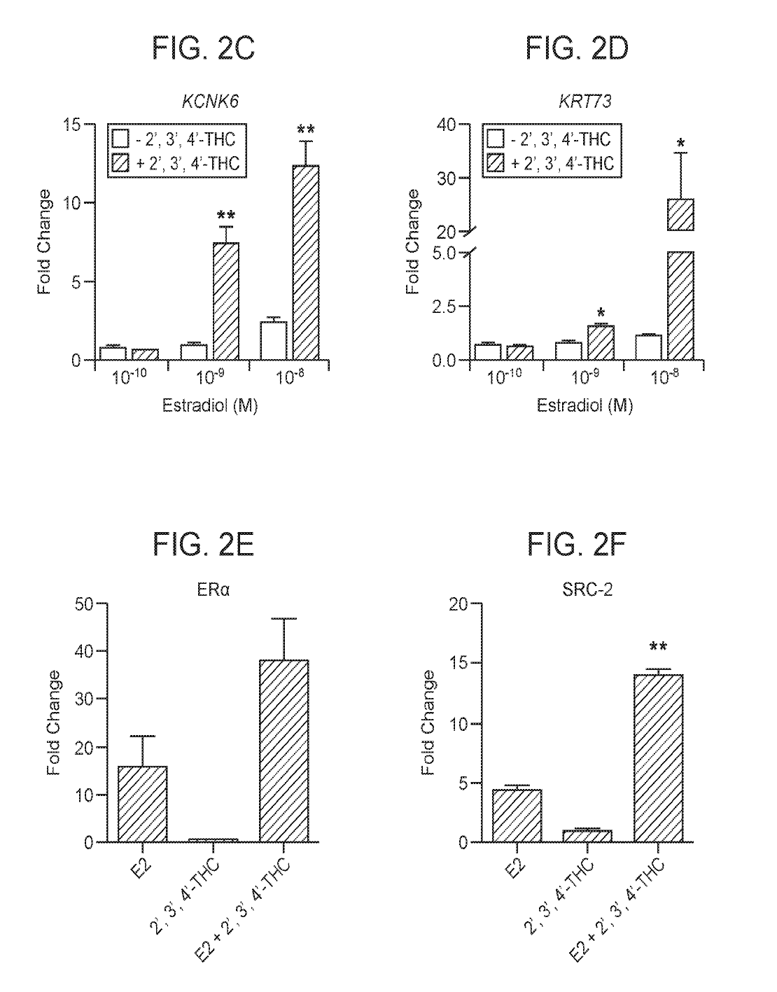

NKG2E, microarray analysis was done in U205-ERα cells. E2 regulated 756 genes, whereas 2′, 3′, 4′-THC alone weakly regulated 31 genes. The 2′, 3′, 4′-THC/E2 combination regulated 1,358 genes, and of these genes 534 were also regulated by E2. 824 genes were termed unique genes because they were only regulated by the 2′, 3′, 4′-THC/E2 combination (FIG. 2a). To verify the microarray results, the activation of the unique genes, FGR (FIG. 2b), KCNK6 (FIG. 2c) and KRT73 (FIG. 2d) were examined KCNK6 and KRT73 were not regulated by E2, whereas an activation of FGR was observed with 10 nM E2. In contrast, the 2′, 3′, 4′-THC/E2 combination induced KCNK6 and KRT73 gene expression at 1 nM, and FGR at 0.1 nM. These findings demonstrate that 2′, 3′, 4′-THC allows E2 to regulate genes at physiological doses. The ERα antagonist ICI 182,780 blocked the induction of KRT73 (FIG. 6a) and NKG2E (FIG. 6b) genes by the 2′, 3′, 4′-THC/E2 combination, demonstrating that ERα is required for gene synergy and unique gene regulation. To explore the mechanism of synergy, whether 2′, 3′, 4′-THC changes the recruitment of ERα and the coactivator SRC-2 at the NKG2E gene was examined The 2′, 3′, 4′-THC/E2 combination was associated with an increased recruitment of ERα (FIG. 2e) and SRC-2 (FIG. 2f) to the NKG2E promoter by chromatin immunoprecipitation (ChIP).

FIGS. 2a-2f2′, 3′, 4′-THC behaves as a unique coagonist on gene expression in U2OS-ERα cells. (FIG. 2a) Venn-diagram showing the total number of genes uniquely regulated by 10 nM E2 alone or 5 μM 2′, 3′, 4′-THC plus E2. E2 uniquely regulated 222 genes. 2′, 3′, 4′-THC with E2 uniquely regulated 824 genes. E2 alone and in combination with 2′, 3′, 4′-THC had 534 genes in common. Expression was assessed by up regulation or down regulation of 3-fold or more and a P-value≤0.05. (FIGS. 2b, 2c, 2d) Gene expression of uniquely regulated genes (FIG. 2b) FGR, (c) KCNK6 and (d) KRT73 in U2OS-ERα cells after treatment with increasing doses of E2 alone or in combination with 5 μM 2′, 3′, 4′-THC for 24 hrs. *P≤0.05, **P≤0.01 compared to E2. (FIGS. 2e, 2f) Chromatin immunoprecipitation of ERα (FIG. 2e) or SRC-2 (FIG. 2f) recruitment to the NKG2E promoter in U2OS-ERα cells after 2 hr treatment with E2 or 2′, 3′, 4′-THC alone or in combination. **P≤0.01 compared to E2. Error bars are ±SEM.

FIGS. 6a-6d The synergistic activation of genes is blocked by the ER antagonist ICI. (FIGS. 6a, 6b) Gene expression of (FIG. 6a) KRT73 and (FIG. 6b) NKG2E in U2OS-ERα cells treated with 10 nM estradiol, 5 μM 2′, 3′, 4′-THC, 1μM ICI alone or in combination for 24 hours. The total cellular RNA was extracted for real-time PCR as described in Materials and Methods. **** P≤0.0001 compared to E2. Error bars are ±SEM. U2OS cells cotransfected with NKG2E-TK-Luc and ERα and treated with 10 nM of E2, 1 μM tamoxifen, 1 μM raloxifene and 5 μM of 2′, 3′, 4′-THC alone or in combination for 24 hours. After transfection, luciferase activities were measured as described in the Methods. RLU is relative light units and the data shown are mean ±SEM. (FIG. 6c). U2OS cells were transfected with NKG2E-TK-Luc and ERα and treated with 5 μM of each chalcone in the absence or presence of 10 nM of E2 for 24 h. After transfection, luciferase activities were measured as described in the Methods. RLU is relative light units and the data shown are mean ±SEM (FIG. 6d).

### Example 4

### 2′, 3′, 4′-THC is a Coligand with E2 that Binds to the ERα LBD

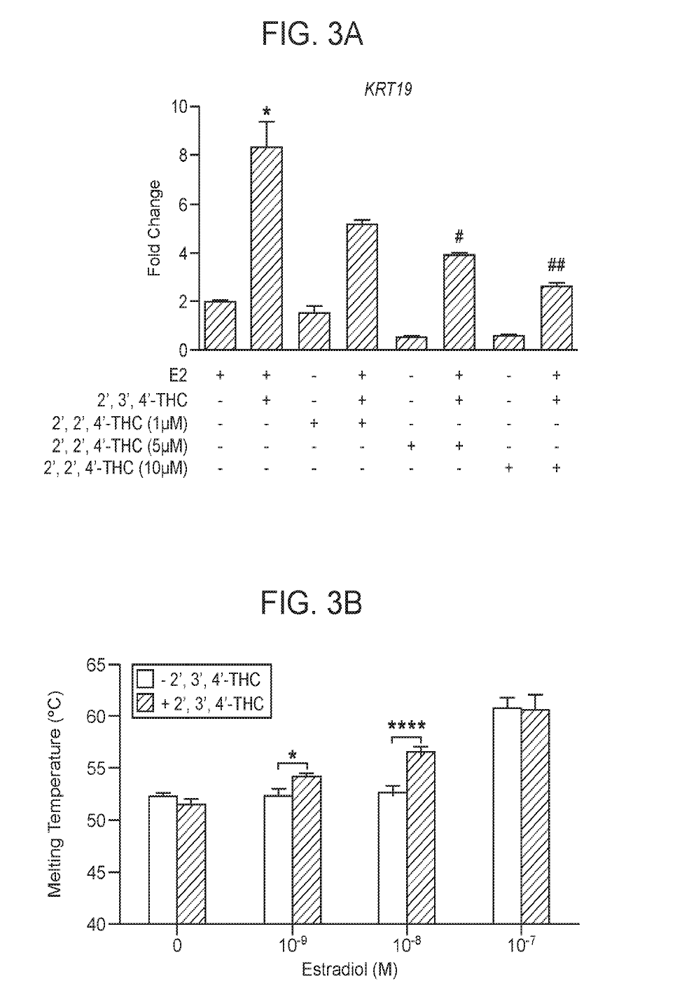

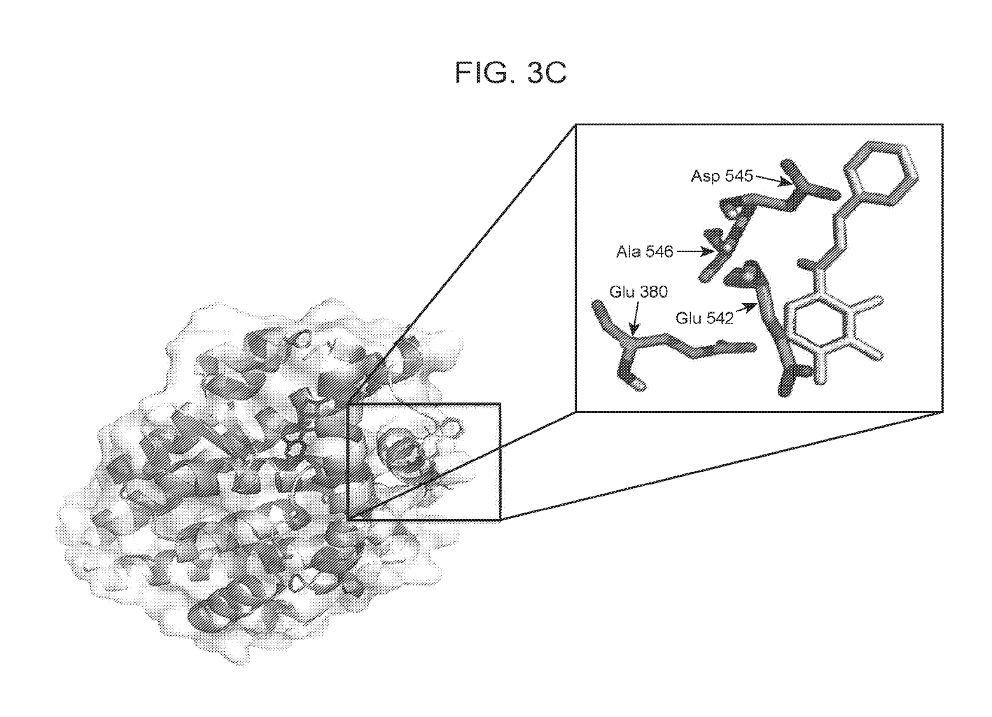

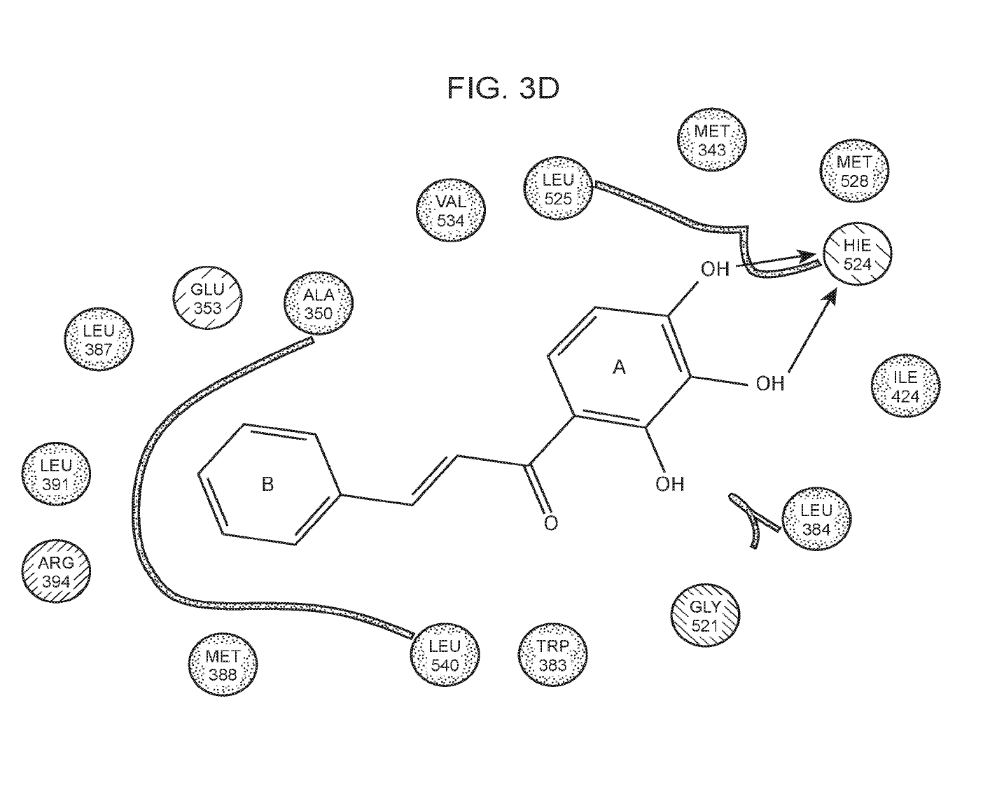

The observation that 2′, 3′, 4′-THC does not compete for E2 binding at concentrations that produce synergy indicates that it does not bind to ERα directly or that it binds to a different site on ERα as a coligand. To explore the hypothesis that 2′, 3′, 4′-THC alters the activity of E2 by forming a coligand with E2 to ERα, another structurally similar chalcone, 2, 2′, 4′-THC found in the screening was tested and was found not to produce a synergistic activation observed with 2′, 3′, 4′-THC. If 2′, 3′, 4′-THC forms a coligand with E2, then 2, 2′, 4′-THC might be able to block the synergy by competing with 2′, 3′, 4′-THC at its binding site, but the E2 activation will be preserved because 2, 2′, 4′-THC will not compete with E2 due to its lower affinity. 2, 2′, 4′-THC blocked the synergistic effect of 2′, 3′, 4′-THC on KRT19 gene expression (FIG. 3a), but had no effect on the activation by E2. To further evaluate a potential formation of a 2′, 3′, 4′-THC coligand with E2, thermostability assays was performed using a purified ERα LBD to measure the melting temperature of the LBD after the addition of the drugs (FIGS. 3b). E2 at 1 and 10 nM did not produce a significant increase in melting temperature, whereas an increase was observed at 100 nM. 2′, 3′, 4′-THC had no effect on the melting temperature of ERα LBD alone, but produced a synergistic increase in the presence of 1 and 10 nM E2. These observations indicate that the change in melting temperature at 1 and 10 nM E2 results from the simultaneous binding of 2′, 3′, 4′-THC and E2 to different sites of ERα . Using the coordinates obtained from the crystal structure previously characterized for E2 complexed to the ERα LBD², in silico computer simulations were performed to determine if there is a potential binding site for 2′, 3′, 4′-THC in ERα . No binding site for 2′, 3′, 4′-THC was found in the cavity where E2 binds, which is consistent the extremely weak competition with [³H]-E2. A potential binding site was identified on the surface of the ERα LBD with four high affinity contact points located in helix 8 (Glu 380) and helix 12 (Glu 542, Asp 545 and Ala 546). When modeling was performed with E2 positioned in its known binding site, 2′, 3′, 4′-THC interacted with a secondary site within the ligand pocket that was adjacent to the E2 binding site (FIG. 3c). 2′, 3′, 4′-THC interacted with amino acids located in helix 3 (Met 343, Glu 353), helix 6 (Leu 384, Leu 387, Leu, 391), helix 11 (Gly 521, Leu 525, His 524, Met 528, and helix 12 (Leu 540) (FIG. 3d). Computer simulations predict that coordinate binding of 2′, 3′, 4′-THC and E2 produces a different conformation of the ERα LBD than with E2 alone. The most dramatic change being a predicted shift in residues 360-370, 460-475, and the N-terminus. The modeling studies indicate that 2′, 3′, 4′-THC binds to ERα as a coligand at a secondary site along with E2.

FIGS. 3a-3d. 2′, 3′, 4′-THC binds to the surface of ERα by in silico modeling. (FIG. 3a) Gene expression of KRT19 after treatment with 10 nM E2, 5 μM 2′, 3′, 4′-THC alone or in combination with increasing doses of 2, 2′, 4′-THC for 24 h. *P≤0.05 compared to control. # P≤0.05, ## P≤0.01 compared to E2/ 2′, 3′, 4′-THC combination. Error bars are ±SEM. (FIG. 3b) Thermostability assays were performed with purified ERα LBD with increasing concentrations of E2 in the absence and presence of 5 μM 2′, 3′, 4′-THC. *P≤0.05, **** P≤0.0001 (FIG. 3c) In silico modeling of 2′, 3′, 4′-THC to the ERα LBD using the coordinates for the X-ray structure with E2. E2 is shown in purple, whereas 2′, 3′, 4′-THC is shown in yellow. 2′, 3′, 4′-THC interacted with amino acids located in helix 3, 6, 11, and 12 (FIG. 3d).

### Example 5

### 2′, 3′, 4′-THC Acts as an ER Antagonist in MCF-7 Breast Cancer Cells

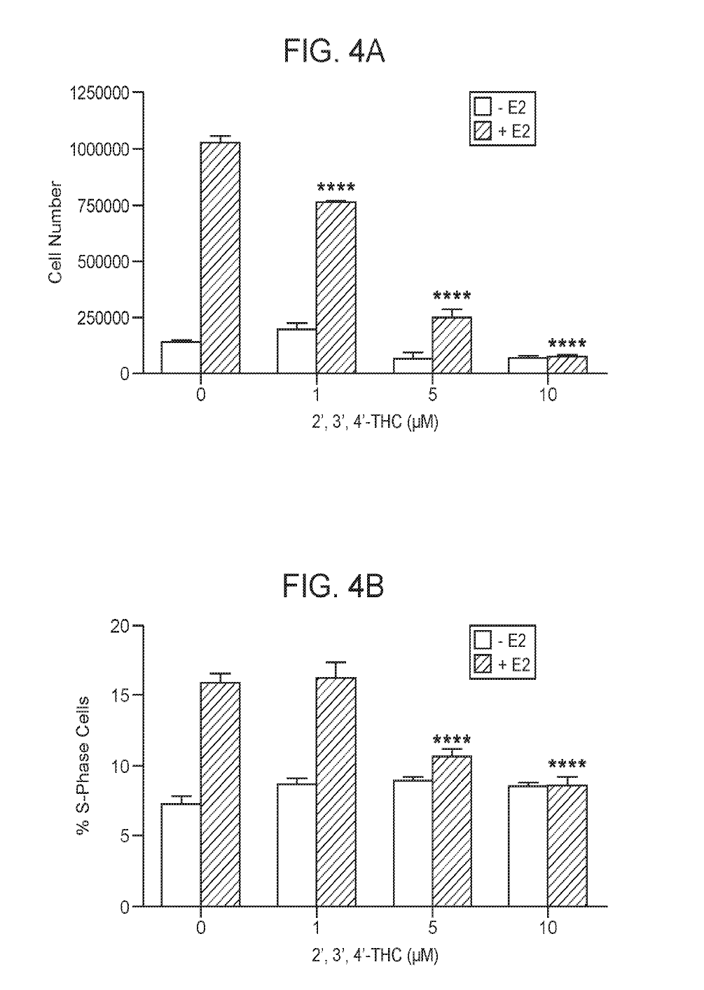

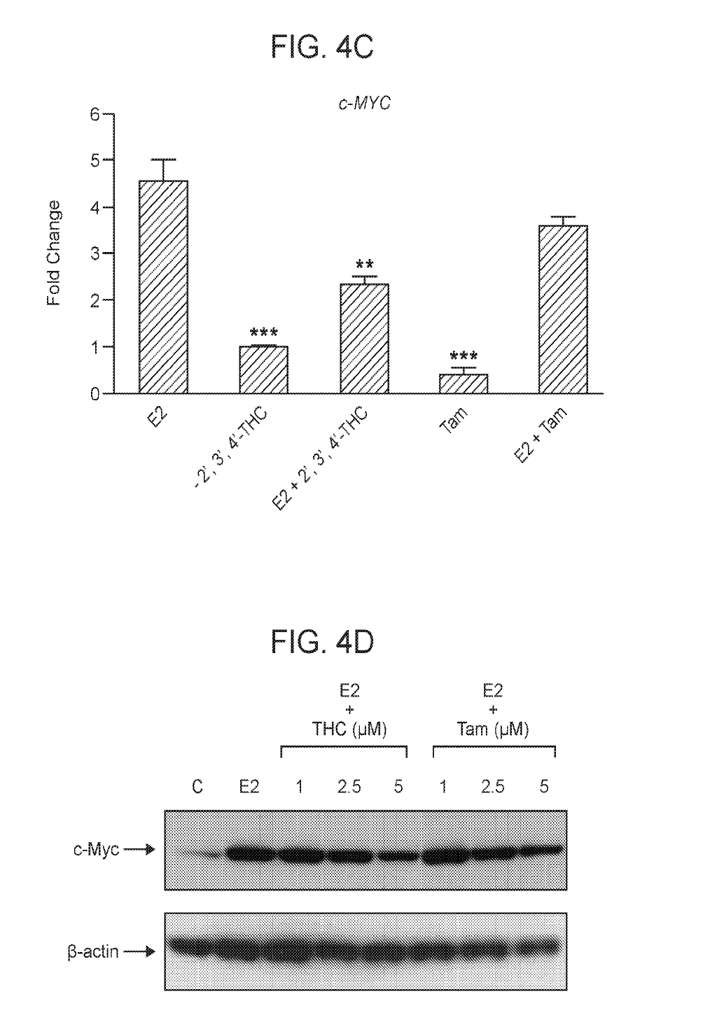

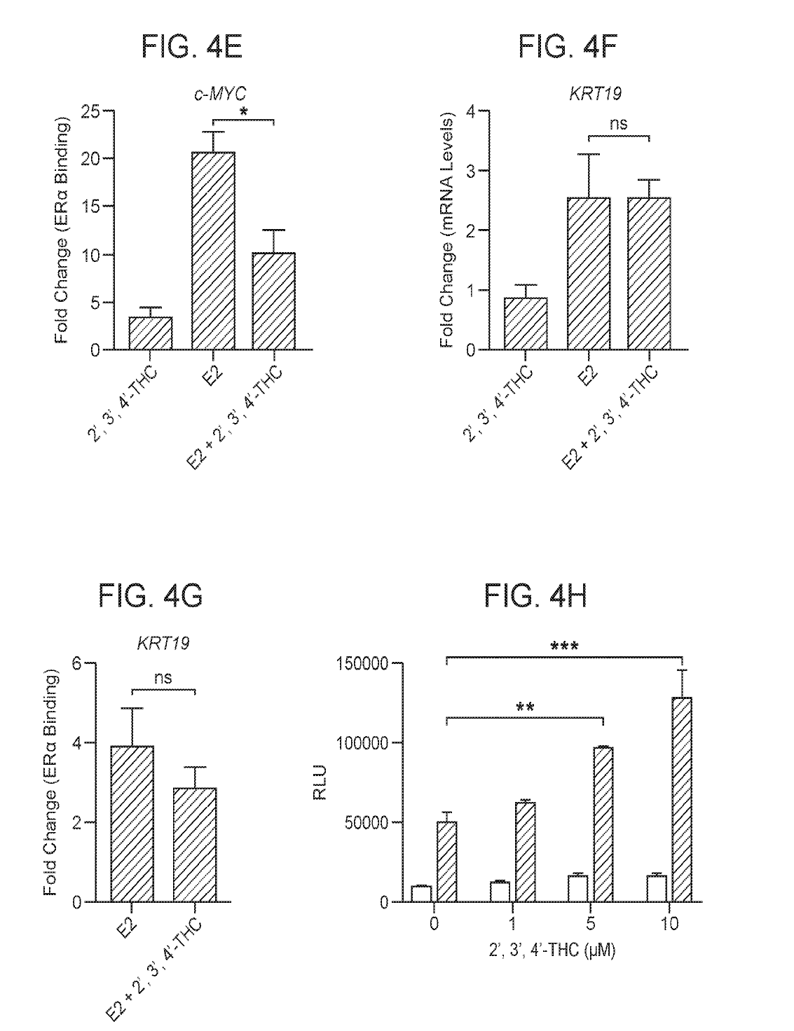

Since ERα promotes proliferation, a synergistic effect of 2′, 3′, 4′-THC on E2 could lead to a greater stimulation of cell proliferation. E2 increased MCF-7 cell proliferation whereas 2′, 3′, 4′-THC alone had no effect on proliferation. The proliferation induced by E2 was blocked by 2′, 3′, 4′-THC in a dose-dependent manner (FIG. 4a). The anti-proliferative action of 2′, 3′, 4′-THC was also observed using flow cytometry to measure DNA content in the cells (FIG. 4b). 2′, 3′, 4′-THC inhibited the E2-stimulation of the oncogene c-MYC mRNA (FIG. 4c) and protein levels (FIG. 4d) greater than tamoxifen. These findings demonstrate that 2′, 3′, 4′-THC inhibits proliferation at doses comparable to tamoxifen in MCF-7 breast cancer cells. To explore the mechanism of the antagonist action of 2′, 3′, 4′-THC in MCF-7 cells, ChIP for ERα recruitment to the c-MYC enhancer, which contains an ERα binding site, was performed. After a 1 h treatment with E2 a significant recruitment of ERα was observed (FIG. 4e). 2′, 3′, 4′-THC inhibited the E2 induced ERα binding to the c-MYC enhancer. In contrast, 2′, 3′, 4′-THC did not have a significant effect on E2 activation of the KRT19 gene (FIG. 4f) or the recruitment of ERα to the KRT19 ERE by ChIP (FIG. 4g). Similar to U2OS cells 2′, 3′, 4′-THC produced a dose-dependent synergistic increase in E2 activation of ERE-TK-Luc in MCF-7 cells (FIG. 4h). Measuring cellular DNA content, the _{IC50.} for tamoxifen inhibition were 3 to 8-fold lower (G1 phase, 0.32 μM; S-phase, 0.98 μM) than 2′, 3′, 4′-THC (G1 phase, 2.9 μM; S-phase, 2.6 μM).

FIGS. 4a-4h 2′, 3′, 4′-THC blocks E2- induced MCF-7 cell proliferation by causing a G1 cell cycle arrest. (FIG. 4a) MCF-7 cell numbers were counted after the cells treated with increasing concentrations of 2′, 3′, 4′-THC with or without E2 (10 nM) for 5 days. (FIG. 4b) Percentage of S phase cells was analyzed with Flow Cytometry after the cells treated with increasing amounts of 2′, 3′, 4′-THC with or without E2 (10 nM) for 24 hours. The data (FIG. 4a) and (FIG. 4b) is expressed as means of triplicate experiments (±SEM). The differences among the samples without and with various amounts of 2′, 3′, 4′-THC in the presence of E2 were analyzed with two-way ANOVA followed by Turkey's multiple comparisons post hoc test. (**** p<0.0001). (FIG. 4c) Levels of c-MYC mRNA were determined by real-time PCR after the cells treated with E2 (10 nM), 2′, 3′, 4′-THC (5 μM), Tamoxifen (Tam) (5 μM), E2+2′, 3′, 4′-THC and E2+Tam for 4 hours. Fold changes were obtained by comparison of control Ct with treated Ct values. The data is expressed as means of biological triplicates ±SEM. The differences among the samples of E2 alone compared to E2+2′, 3′, 4′-THC and E2+Tam were analyzed with one-way ANOVA followed by Turkey's multiple comparisons post hoc test. (** p<0.01; *** p<0.001). (FIG. 4d) Western blot of c-MYC Protein levels using a monoclonal anti-cMYC IgG was carried out after the cells treated with E2 (10 nM) and increasing doses of 2′, 3′, 4-THC (THC) or Tamoxifen (Tam) for 1 hour. (FIG. 4e) ERα recruitment to c-MYC enhancer region was analyzed with ChIP using a rabbit ERα antibody after the cells were treated with E2 (10 nM) in the absence or presence of 2′, 3′, 4-THC (5 μM) for 1 hour. The data shown is derived from qPCR analysis of an ERα binding site on c-MYC enhancer region. The Ct values of ERα antibody precipitated DNA were adjusted by corresponding input value. The fold changes were obtained by comparison of adjusted Ct values of treated samples with non-treated sample (control). The results are expressed as mean±SEM from triplicate experiments. The difference among various treatments was analyzed with One-way ANOVA followed by Turkey's multiple comparisons post hoc test. (* p<0.05). (FIG. 4f) KRT19 mRNA was determined by reverse transcription-quantitative polymerase chain reaction (RT-QPCR) after the cells treated with E2 (10 nM), 2′, 3′, 4′-THC (5 μM), and E2+2′, 3′, 4′-THC for 4 hours in serum-free DMEM. Fold changes were obtained by comparison of control Ct value to treated Ct values. The data is expressed as means of biological triplicates ±SEM. The statistical analysis is the same as for (FIG. 4c). (FIG. 4g) ERα recruitment to KRT19 ERE analyzed by ChIP. MCF-7 cells were treated and subjected to ChIP assay the same as for panel (e). The data shown are derived from qPCR analysis of an ERα binding site on the KRT19 ERE. The data was analyzed the same as for (FIG. 4e). MCF-7 cells were transfected ERE-TK-Luc and then treated with 1, 5 or 10 μM of 2′, 3′, 4′-THC in absence or presence of 10 nM of E2. Luciferase activity was measured as described in the Methods (FIG. 4h).

### Example 6

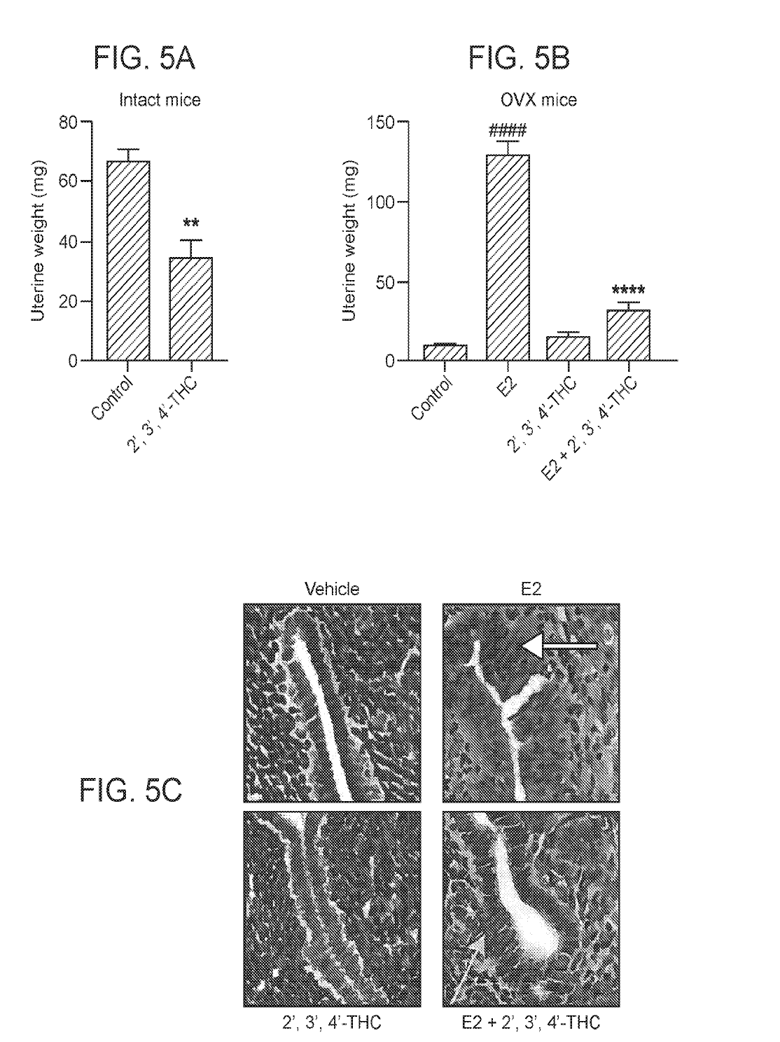

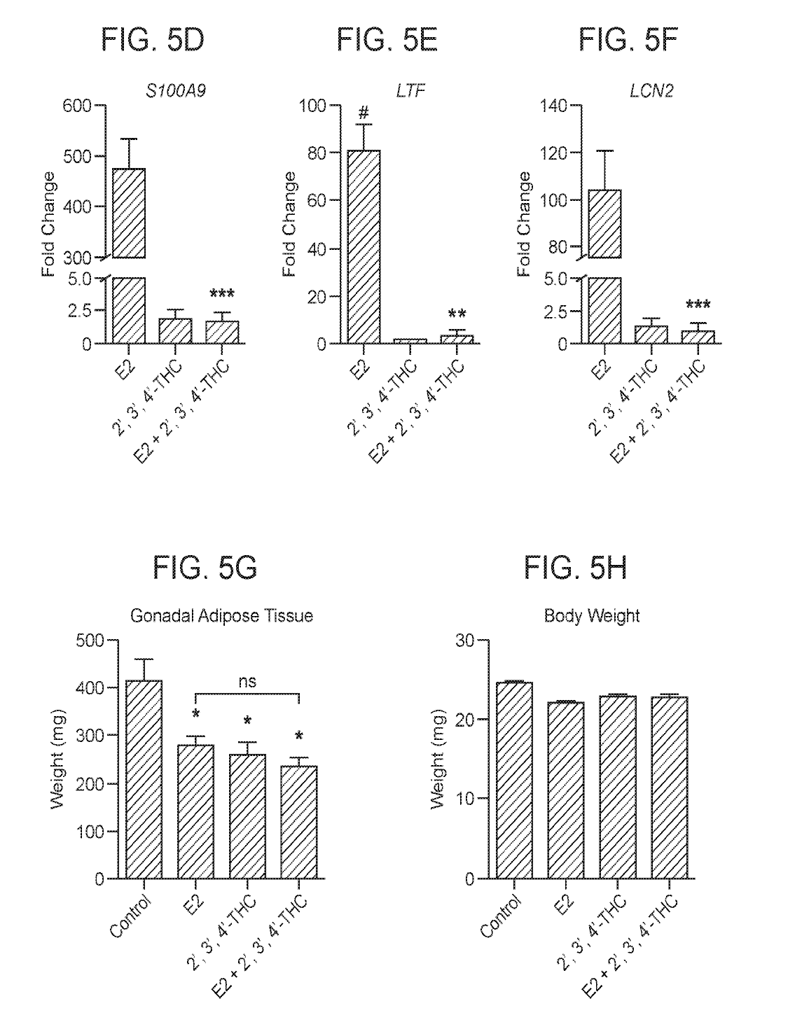

### 2′, 3′, 4′-THC Acts as an ERα Antagonist in the Mouse Uterus

Whereas 2′, 3′, 4′-THC blocked the proliferative effects of E2 on MCF-7 cells it is conceivable that 2′, 3′, 4′-THC could enhance the proliferative effects in the uterus by endogenous estrogens if it acts synergistically with E2 in vivo. Intact C57BL/6 female mice were treated with 2′, 3′, 4′-THC for 4 weeks and the size of the uterus was measured. Mice treated with 2′, 3′, 4′-THC had a 50% reduction of uterine growth compared to the control mice, suggesting that 2′, 3′, 4′-THC antagonizes the effect of endogenous estrogens on the uterus (FIG. 5a). In an ovariectomized (OVX) mouse model, 2′, 3′, 4′-THC did not increase uterine size, whereas E2 produced a large increase in uterine weight (FIG. 5b). The E2-induced increase in uterine weight was blocked by 2′, 3′, 4′-THC (FIG. 5b). H & E staining showed that E2 increased the number of cells lining the duct (FIG. 5c, upper right panel) and that 2′, 3′, 4′-THC inhibited the proliferative response (FIG. 5c, lower right panel). The antagonist effect of 2′, 3′, 4′-THC in the uterus was observed on E2-induced increased levels of mRNA for SA100A9 (FIG. 5d), LTF (FIG. 5e) and LCN2 (FIG. 5f), which are known target genes for E2¹⁷. In contrast, E2 decreased the amount of gonadal adipose tissue fat in mice and this was not blocked by 2′, 3′, 4′-THC (FIG. 5g). No significant change in body weight was observed with the different treatments (FIG. 5h). These findings demonstrate that 2′, 3′, 4′-THC acts as an E2 antagonist in the mouse uterus, but did not prevent the decrease in gonadal adipose tissue by E2.

FIGS. 5a-5h. 2′, 3′, 4′-THC acts as antagonist in the mouse uterus. Uterine weights of intact (FIG. 5a) and OVX (FIG. 5b) mice. (FIG. 5a) Intact mice received a daily intraperitoneal injection of 2′, 3′, 4′-THC treated for 3 weeks and the uterus was removed and weighed. **P≤0.01 compared to vehicle treated mice (FIG. 5b) OVX mice were treated with 2′, 3′, 4′-THC or in combination with E2 using an osmotic pump for 4 weeks. **P≤0.01, **** P≤0.0001, compared to E2. #### P≤0.0001, compared to vehicle control (n=5/group). (FIGS. 5d, 5e, 5f) Gene expression in uterine tissue of OVX mice treated with E2 alone or in combination with 2′, 3′, 4′-THC for 4 weeks. #P≤0.05 compared to vehicle control. **P≤0.01, *** P≤0.001 compared to E2 group. (FIG. 5g) Gonadal adipose tissue weights of OVX mice treated with 2′, 3′, 4′-THC alone or in combination with E2 for 4 weeks. (FIG. 5h) Total body weight of OVX mice after 4 week treatment. Error bars are ±SEM.

While the present disclosure has been described with reference to the specific embodiments thereof, it should be understood by those skilled in the art that various changes may be made and equivalents may be substituted without departing from the true spirit and scope of the present disclosure. In addition, many modifications may be made to adapt a particular situation, material, composition of matter, process, process step or steps, to the objective, spirit and scope of the present disclosure. All such modifications are intended to be within the scope of the claims appended hereto.

What is claimed is:

1. A method of treating an individual for an estrogen receptor (ER)-associated condition, comprising administering a therapeutically effective amount of an ERα coligand to an individual, wherein the ERα coligand is a cell type-selective, allosteric modulator of ERα signaling, thereby reprogramming ER agonist-dependent signaling in a tissue-selective manner

2. The method of claim 1, wherein the reprogramming comprises:
suppressing ER agonist-dependent cell proliferation in breast and/or uterine tissue, relative to ER agonist-dependent cell proliferation in the absence of the ERα coligand; and/or
increasing ER agonist-dependent transcription in bone, brain and/or adipose tissue, relative to ER agonist-dependent signaling in the absence of the ERα coligand.

3. The method of any one of claims 1 and 2, wherein the ERα coligand is a compound, or a pharmaceutically acceptable salt thereof, represented by the formula (I): 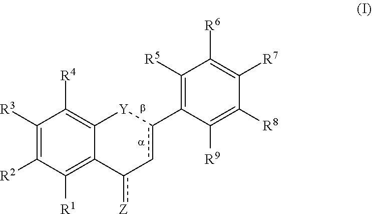
wherein
α, β and γ are optional bonds, with the proviso that when β is absent, a is present;
R¹, R², R³, R⁴, R⁵, R⁶, R⁷, R⁸, and R⁹ are independently hydrogen, hydroxyl, sulfhydryl, halo, alkyl, alkoxy, aryloxy, or arylalkyloxy, and may be heteroatom-containing and/or substituted;
Y is hydrogen, hydroxyl, sulfhydryl, halo, alkyl, alkoxy, aryloxy, or arylalkyloxy, when is absent, and O, when is present; and
Z is O, S or NR¹⁰, where R¹⁰ is hydrogen or alkyl.

4. The method of claim 3, wherein the ERα coligand has a molecular weight in the range of 250 to 260 g/mol.

5. The method of any one of claims 3 and 4, wherein α and γ are present.

6. The method of any one of claims 3 to 5, wherein R⁵, R⁶, R⁷, R⁸, and R⁹ are hydrogen.

7. The method of any one of claims 5 and 6, wherein the ERα coligand is a chalcone derivative.

8. The method of claim 7, wherein the ERα coligand is a trihydroxychalcone.

9. The method of claim 8, wherein the ERα coligand is 2′,3′,4′-trihydroxychalcone (2′,3′, 4′-THC).

10. The method of any one of claims 1 to 9, wherein the ERα coligand is administered at an amount sufficiently low to achieve a local concentration of the ERα coligand in a target tissue at which local concentration the coligand does not substantially compete with an ER agonist for binding to the estrogen binding site of ERα in the target tissue.

11. The method of any one of claims 1 to 10, wherein the ERα coligand modulates ERβ signaling.

12. The method of claim 11, wherein the ERα coligand increases ERβ signaling relative to ERβ signaling in the absence of the ERα coligand.

13. The method of any one of claims 1 to 12, wherein the administering comprises parenterally or orally administering the ERα coligand to the patient.

14. The method of any one of claims 1 to 13, wherein the method further comprises co-administering a pharmaceutically effective amount of an ER agonist with the ERα coligand.

15. The method of claim 14, wherein the ER agonist is estradiol (E2), or a derivative thereof.

16. The method of any one of claims 14 and 15, wherein the method does not comprise co-administering a progestin with the ER agonist.

17. The method of any one of claims 14 to 16, wherein the molar ratio of the ER agonist to the ERα coligand administered to the individual is 1:100 or less.

18. The method of any one of claims 1 to 17, wherein allosteric modulation of ERα signaling by the ERα coligand is inhibited by 2,2′, 4′-THC at a concentration of 2,2′, 4′-THC that does not inhibit ER agonist-dependent signaling.

19. The method of any one of claims 1 to 18, wherein the ER-associated condition comprises symptoms of menopause, side effects of menopausal hormone therapy and/or cancer.

20. The method of claim 19, wherein the ER-associated condition comprises osteoporosis, breast cancer, endometrial cancer, colon cancer, pulmonary cancer, dementia, Alzheimer's disease, hot flashes, mood swings, insomnia, vaginal atrophy, vaginal dryness, dyspareunia, venous thromboembolism, gallbladder disease, obesity and/or diabetes.

21. The method of any one of claims 1 to 20, wherein the individual is a pre-, peri- or post-menopausal individual.

22. A pharmaceutical composition comprising a pharmaceutically effective amount of an ERα coligand in a pharmaceutically acceptable excipient, wherein the ERα coligand is a cell type-selective, allosteric modulator of ER signaling.

23. The composition of claim 22, wherein the ERα coligand suppresses ER agonist-dependent cell proliferation in breast and/or uterine tissue relative to ER agonist-dependent cell proliferation in the absence of the ERα coligand, and increases ER agonist-dependent signaling in bone, brain and/or adipose tissue relative to ER agonist-dependent signaling in the absence of the ERα coligand.

24. The composition of claim 22 or 23, wherein the ERα coligand is a chalcone derivative.

25. The composition of claim 24, wherein the ERα coligand is a trihydroxychalcone, or a pharmaceutically acceptable salt thereof.

26. The composition of claim 25, wherein the ERα coligand is 2′, 3′, 4′-THC.

27. The composition of any one of claims 22 to 26, wherein the composition further comprises a pharmaceutically effective amount of an ER agonist.

28. The composition of claim 27, wherein the molar ratio of the ER agonist to the ERα coligand is 1:100 or less.

29. The composition of any one of claims 22 to 28, wherein the ERα coligand modulates ERβ signaling.

30. The composition of claim 29, wherein the ERα coligand increases ERβ signaling relative to ERβ signaling in the absence of the ERα coligand.
